SEQUENCE LISTING (34 sequences)

SEQ ID NO: 1 - protein, 14 aa, Homo sapiens
FSSYAMSWVRQAPG

SEQ ID NO: 2 - protein, 19 aa, Homo sapiens
SAISGSGGSTYYADSVKGR

SEQ ID NO: 3 - protein, 21 aa, Homo sapiens
ARRKKRERGFSGNDPVGAIDV

SEQ ID NO: 4 - protein, 15 aa, Homo sapiens
CTGSSSNIGAGYDVH

SEQ ID NO: 5 - protein, 7 aa, Homo sapiens
GNTNRPS

SEQ ID NO: 6 - protein, 12 aa, Homo sapiens
CSAWDDSLSGVV

SEQ ID NO: 7 - protein, 128 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRKKRERGFSGNDPVGAIDVWGQGTLVTVSS

SEQ ID NO: 8 - protein, 112 aa, Homo sapiens
QSVLTQPPSASGTPGQRVTISCTGSSSNIGAGYDVHWYQQLPGTAPKLLIYGNTNRPSGVPDRFSGSKSGTSASLAISGLRSEDEADYYCSAWDDSLSGVVFGGGTKLTVLG

SEQ ID NO: 9 - protein, 14 aa, Homo sapiens
FSSYWMSWVRQAPG

SEQ ID NO: 10 - protein, 19 aa, Homo sapiens
SRINTHGTNIDYADSVKGR

SEQ ID NO: 11 - protein, 11 aa, Homo sapiens
VGVAGTGWFDP

SEQ ID NO: 12 - protein, 15 aa, Homo sapiens
CTGSSSNIGAGYDVH

SEQ ID NO: 13 - protein, 7 aa, Homo sapiens
GNNNRPS

SEQ ID NO: 14 - protein, 12 aa, Homo sapiens
CQSYDTSLSGSV

SEQ ID NO: 15 - protein, 118 aa, Homo sapiens
EVQLLESGGGLVQPGGSLRLSCAASGFTFSSYWMSWVRQAPGKGLEWVSRINTHGTNIDYADSVKGRFTISRDNSKNTLYLQMNSLRAEDTAVYYCVGVAGTGWFDPWGQGTLVTVSS

SEQ ID NO: 16 - protein, 112 aa, Homo sapiens
QSVLTQPPSASGTPGQRVTISCTGSSSNIGAGYDVHWYQQLPGTAPKLLIYGNNNRPSGVPDRFSGSKSGTSASLAISGLRSEDEADYYCQSYDTSLSGSVFGGGTKLTVLG

SEQ ID NO: 17 - protein, 14 aa, Homo sapiens
FSSYSMNWVRQAPG

SEQ ID NO: 18 - protein, 19 aa, Homo sapiens
SAISGSGGSTYYADSVKGR

SEQ ID NO: 19 - protein, 11 aa, Homo sapiens
ARGLATYGLDV

SEQ ID NO: 20 - protein, 14 aa, Homo sapiens
CSGSSSNIGRHHVY

SEQ ID NO: 21 - protein, 7 aa, Homo sapiens
SNSLRPS

SEQ ID NO: 22 - protein, 12 aa, Homo sapiens
CAAWDDSLSGWV

SEQ ID NO: 23 - protein, 118 aa, Homo sapiens
EVQLLESGGGLVQPGGSLRLSCAASGFTFSSYSMNWVRQAPGKGLEWVSAISGSGGSTYYADSVKGRFTISRDNSKNTLYLQMNSLRAEDTAVYYCARGLATYGLDVWGQGTLVTVSS

SEQ ID NO: 24 - protein, 111 aa, Homo sapiens
QSVLTQPPSASGTPGQRVTISCSGSSSNIGRHHVYWYQQLPGTAPKLLIYSNSLRPSGVPDRFSGSKSGTSASLAISGLRSEDEADYYCAAWDDSLSGWVFGGGTKLTVLG

SEQ ID NO: 25 - protein, 14 aa, Homo sapiens
FSSYSMNWVRQAPG

SEQ ID NO: 26 - protein, 19 aa, Homo sapiens
ANIKQDGTENYYVDSVEGR

SEQ ID NO: 27 - protein, 12 aa, Homo sapiens
ARDGDWGWGFDY

SEQ ID NO: 28 - protein, 15 aa, Homo sapiens
CTGSSSNIGAGYDVH

SEQ ID NO: 29 - protein, 7 aa, Homo sapiens
ENNKRPS

SEQ ID NO: 30 - protein, 12 aa, Homo sapiens
CAAWDDSLSGWV

SEQ ID NO: 31 - protein, 119 aa, Homo sapiens
EVQLLESGGGLVQPGGSLRLSCAASGFTFSSYSMNWVRQAPGKGLEWVANIKQDGTENYYVDSVEGRFTISRDNSKNTLYLQMNSLRAEDTAVYYCARDGDWGWGFDYWGQGTLVTVSS

SEQ ID NO: 32 - protein, 112 aa, Homo sapiens
QSVLTQPPSASGTPGQRVTISCTGSSSNIGAGYDVHWYQQLPGTAPKLLIYENNKRPSGVPDRFSGSKSGTSVSLAISGLRSEDEADYYCAAWDDSLSGWVFGGGTKLTVLG

SEQ ID NO: 33 - protein, 330 aa, Homo sapiens [sequence omitted]

SEQ ID NO: 34 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0070339 A1
(43) Pub. Date: Mar. 9, 2023

(54) LILRB3 ANTIBODY MOLECULES AND USES THEREOF

(71) Applicants: University of Southampton, Southampton Hampshire (GB); BIOINVENT INTERNATIONAL AB, Lund (SE)

(72) Inventors: Björn FRENDÉUS, Lund (SE); All Roghanian, Southampton Hampshire (GB); Mark GRAGG, Southampton Hampshire (GB)

(21) Appl. No.: 17/799,240

(22) PCT Filed: Feb. 12, 2021
(86) PCT No.: PCT/EP2021/053516

§ 371 Date: Aug. 11, 2022

(30) Foreign Application Priority Data
Feb. 12, 2020 (EP) 20156969.6

(51) Int. Cl. C07K 16/28; A61P 37/06

(52) U.S. Cl. CPC C07K 16/2803; A61P 37/06; A61K 2039/505

(57) ABSTRACT

Described are anti-LILRB3 antibody molecules, such as agonistic anti-LILRB3 antibody molecules for use in treatment of graft rejection or autoimmunity via reprograming of human myeloid cells. Described are also specific anti-LILRB3 antibody molecules and use of such antibody molecules in medicine, for example in treatment of graft rejection, autoimmune disorders or inflammatory disorders.

### FIELD OF THE INVENTION

The present invention relates to novel antibody molecules that specifically bind to LILRB3 (ILT5). The invention also relates to the use of such novel antibody molecules or other antibody molecules that specifically bind to LILRB3 (ILT5) in treatment of graft rejection, an autoimmune disorder and/or an inflammatory disorder.

### BACKGROUND OF THE INVENTION

The family of human leukocyte immunoglobulin (Ig)-like receptors (LILRs), also called human immunoglobulin-like transcripts (ITLs), comprises six activating (LILRA1-6) and five inhibitory (LILRB1-5) LILRs that regulate immune responses (1, 2). Both receptor subtypes display two, or four, homologous C-2 type immunoglobulin (Ig)-like extracellular domains, but differ in their transmembrane and cytoplasmic regions (3, 4). LILRAs have short truncated cytoplasmic domains with charged arginine residues in their transmembrane domains, allowing them to associate with the γ-chain of ITAM-bearing FcεR to propagate activating signaling cascades (5). Conversely, LILRB have long cytoplasmic domains that contain multiple ITIM domains, which recruit phosphatases such as SHP-1 and SHIP-1 that elicit inhibitory signaling (3, 4). Located at human chromosome 19q13.4, these polygenic receptors demonstrate significant allelic variation, with LILRB3 (ILT5/CD85a) and LILRB4 (ILT3/CD85k) displaying at least 15 different variants (3. 6).

The inhibitory LILRBs are proposed to act as immune checkpoints serving to control and limit overt immune responses (1, 2). In agreement with this, LILRB expression is increased in suppressive (also referred to as alternatively activated or M2) macrophages and tolerogenic dendritic cells (DCs) (7-10). On monocytes, co-ligation of LILRB1 (ILT2/CD85j) and LILRB2 (ILT4/CD85d) with FcγRI (CD64) results in SHP-1 activation, decreasing downstream phosphorylation events and intracellular calcium mobilization (11). Upon ligation with HLA class I (HLA-I) ligands, LILRB1 and LILRB2 prevent migration of DCs, and promote their release of anti-inflammatory cytokines (1, 12). Similarly, engagement of LILRB1 on macrophages by the common HLA-I subunit β2-microglobulin on malignant cells limits their phagocytic potential (13). LILRBs have also been shown to render DCs tolerogenic both in vitro and in vivo, subsequently inhibiting T cell responses (7, 8, 12, 14, 15). As such, the engagement of HLA-G with LILRB1 and LILRB2 is an important immunosuppressive pathway at the fetal-maternal interface during pregnancy (16-18). LILRB1 is also expressed on NK cells and has been reported to inhibit NK cell cytotoxicity (19).

Although mice do not express LILRBs, the orthologous paired Ig-like receptor (PIR)-B regulates various arms of the immune system. PIR-B regulates priming of cytotoxic T-lymphocytes by DCs via interaction with MHC class I expressed on CD8 cells (20); and negatively influences integrin signaling in neutrophils and macrophages (21). Furthermore, PIR-B regulates the differentiation of myeloid-derived suppressor cells (MDSCs) that aid in tumor progression (22). Similar to PIR-B, the interaction between HLA-G and LILRB1 supports allotransplant engraftment through expansion of potent MDSC (23, 24).

Among the inhibitory LILRBs, LILRB3 (ILT5/LIR3/CD85a), containing 4 intracellular ITIM motifs, presents an attractive immunomodulatory target due to its relative restriction to, and high expression on, myeloid cells (2). Despite its discovery in the late 1990's, its exact functions and immunomodulatory potential have not been fully determined, due to the lack of specific reagents and model systems

### SUMMARY OF THE INVENTION

To investigate the potential immunomodulatory capacity of LILRB3, a panel of LILRB3-specific monoclonal antibodies (mAb) was generated using BioInvent International AB's proprietary n-CoDeR® and F.I.R.S.T™ platform technology. The antibodies bound to two major but discrete epitopes in Ig-like domains 2 and 4. LILRB3 ligation on primary human monocytes and macrophages resulted in phenotypic and functional changes and potent inhibition of immune responses in vitro, including significant reduction in phagocytosis of opsonized cancer cells and T cell proliferation. Importantly, targeting of LILRB3 in humanized mice induced a tolerogenic status and permitted enhanced engraftment of allogeneic human lymphoma cells. Our findings reveal immunoregulatory functions of human LILRB3 and identify its potential as an important myeloid immune checkpoint, with potential roles in transplantation, infection and autoimmunity.

The work leading to the present invention comprised the following:
generation and characterization of a panel of human monoclonal anti-LILRB3 antibodies agonistic activity,
demonstrating that ligation of LILRB3 on human myeloid cells induces an anti-inflammatory phenotype, leading to subsequent inhibition of T cell proliferation
demonstrating that LILRB3 ligation on human macrophages inhibits phagocytosis of opsonized target cells
demonstrating that agonistic anti-LILRB3 antibodies induced tolerance in humanized mice, permitting successful engraftment of allogeneic cells.

Thus, the present invention relates to antibody molecules that bind specifically to LILRB3 (ILT5) for use in treatment of graft rejection, autoimmune disorders and/or inflammatory disorders.

The present invention also relates to specific antibody molecules that bind specifically to LILRB3 (ILT5) selected from the group consisting of antibody molecules comprising 1-6 of the CDRs VH-CDR1, VH-CDR2, VH-CDR3, VL-CDR1, VL-CDR2 and VL-CDR3,

wherein VH-CDR1, if present, is selected from the group consisting of SEQ. ID. NOs: 1, 9, 17 and 25;

wherein VH-CDR2, if present, is selected from the group consisting of SEQ. ID. NOs: 2, 10, 18 and 26;

wherein VH-CDR3, if present, is selected from the group consisting of SEQ. ID. NOs: 3, 11 and 19 and 27;

wherein VL-CDR1, if present, is selected from the group consisting of SEQ. ID. NOs: 4, 12, 20 and 28;

wherein VL-CDR2, if present, is selected from the group consisting of SEQ. ID. NOs: 5, 13, 21 and 29; and

wherein VL-CDR3, if present, is selected from the group consisting of SEQ. ID. NOs: 6, 14, 22 and 30.

The present invention also relates to isolated nucleotide sequences encoding at least one of the above antibody molecules.

The present invention also relates to plasmids comprising at least one of the above nucleotide sequences.

The present invention also relates to cells comprising at least one of the above nucleotide sequences, or at least one of the above plasmids.

The present invention also relates to the above antibody molecules, nucleotide sequences, plasmids and/or cells for use in medicine.

The present invention also relates to the above antibody molecules, nucleotide sequences, plasmids and/or cells for use in the treatment of graft rejection.

The present invention also relates to the above antibody molecules, nucleotide sequences, plasmids and/or cells for use in the treatment of an autoimmune disorder (also denoted autoimmunity).

The present invention also relates to the above antibody molecules, nucleotide sequences, plasmids and/or cells for use in the treatment of an inflammatory disorder.

The present invention also relates to the use of the above antibody molecules, nucleotide sequences, plasmids and/or cells for use in the treatment of graft rejection.

The present invention also relates to the use of the above antibody molecules, nucleotide sequences, plasmids and/or cells for use in the treatment of an autoimmune disorder.

The present invention also relates to the use of the above antibody molecules, nucleotide sequences, plasmids and/or cells for use in the treatment of an inflammatory disorder.

The present invention also relates to pharmaceutical compositions comprising or consisting of at least one of the above antibody molecules, nucleotide sequences, plasmids and/or cells, and optionally a pharmaceutically acceptable diluent, carrier, vehicle and/or excipient. Such a pharmaceutical composition may be used in the treatment of graft rejection. Such a pharmaceutical composition may also or alternatively be used in the treatment of an autoimmune disorder. Such a pharmaceutical composition may also or alternatively be used in the treatment of an inflammatory disorder.

The present invention also relates to methods for treatment of graft rejection in a patient comprising administering to the patient a therapeutically effective amount of at least one of the above antibody molecules, nucleotide sequences, plasmids and/or cells.

The present invention also relates to methods for treatment of an autoimmune disorder in a patient comprising administering to the patient a therapeutically effective amount of at least one of the above antibody molecules, nucleotide sequences, plasmids and/or cells.

The present invention also relates to methods for treatment of an inflammatory disorder in a patient comprising administering to the patient a therapeutically effective amount of at least one of the above antibody molecules, nucleotide sequences, plasmids and/or cells.

The present invention also relates to antibody molecules, antibody molecules for use, isolated nucleotide sequences, isolated nucleotide sequences for use, plasmids, plasmids for use, cells, cells for use, uses, pharmaceutical compositions and methods of treatment as described herein with reference to the accompanying description, examples and/or figures.

### DETAILED DESCRIPTION OF THE INVENTION

Thus, the present invention concerns antibody molecules that bind specifically to LILRB3 (ILT5). In this context, the term “antibody molecule that specifically binds LILRB3” can be used interchangeably with the term “anti-LILRB3 antibody molecule (or “antibody molecule that specifically binds ILT5” and “anti-ILT5 antibody molecule, respectively) refers to an antibody molecule that specifically binds to at least one epitope in the extracellular domain of LILRB3 (ILT5). Cell surface antigen and epitope are terms that would be readily understood by one skilled in immunology or cell biology.

Methods of assessing protein binding are known to the person skilled in biochemistry and immunology. It would be appreciated by the skilled person that those methods could be used to assess binding of an antibody to a target; as well as the relative strength, or the specificity, or the inhibition, or prevention, or reduction in those interactions. Examples of methods that may be used to assess protein binding are, for example, immunoassays, Biacore, western blots, radioimmunoassay (RIA) and enzyme-linked immunosorbent assays (ELISAs) and Flow cytometry (FACS). See Fundamental Immunology Second Edition, Raven Press, New York at pages 332-336 (1989) for a discussion regarding antibody specificity.

The target cells expressing the LILRB3 to which the antibody molecule bind in accordance with the present invention can be any LILRB3 expressing cells, such as human myeloid cells, including monocytes and macrophages.

Without being bound to any specific mechanism, one hypothesis is that the effect of the binding of the antibody molecules according to the invention to LILRB3 may be that it leads to the phosphorylation of the ITIM domains. LILRB3 contains four intracellular ITIMs. This, in turn, inhibits cellular activation and induces the production of immunosuppressive genes by the myeloid cells. This is evident from the example below showing RNAseq analysis of human monocytes.

In some embodiments, the agonistic activity may be improved by the antibody molecule binding to an Fcγ receptor in addition to binding to LILRB3. In some such embodiments, the agonistic non-blocking LILRB3 antibody molecules bind with higher affinity to inhibitory Fcγ receptors than to activating Fcγ receptors. With higher affinity to inhibitory Fcγ receptors than to activating Fcγ receptors, we include the meaning of variants that bind with higher affinity to inhibitory Fcγ receptors compared with individual activating Fcγ receptors, e.g. compared with either of FcγRIIA, FcγRIIIA and FcγRI.

The relatively high homology between mouse and human FcγR systems accounts for many of the general aspects of conserved FcγR mediated mechanisms between the species. However, mouse and human IgG subclasses differ in their affinities for their cognate FcγRs, making it important when translating FcγR-mediated observations in the mouse system into human IgG-based therapeutics to choose an antibody, antibody subclass and/or engineered subclass variant, that shows appropriate binding to human activating vs inhibitory FcγRs. The affinity and/or avidity of human antibody molecules for individual human FcγRs can be determined using surface plasmon resonance (SPR).

In some embodiments, the binding to an Fc receptor occurs through normal interaction between the Fc region of the agonistic antibody molecule and the Fc receptor. In some such embodiments the antibody molecule is an IgG, which has an Fc region binding to an Fcγ receptor. In some such embodiments, the anti-LILRB3 antibody is of human IgG2 isotype, which has similar intermediate affinity for human inhibitory FcγRIIB and human activating FcγRIIA and FcγRIIIA, but does not productively engage with human activating FcγRI. In some embodiments the anti-LILRB3 antibody is of human IgG1 isotype, which binds FcγRIIB with higher affinity compared with IgG2, but also binds activating human activating FcγRIIA, FcγRIIIA with higher affinity, and additionally binds activating FcγRI with high affinity. In other embodiments, the anti-LILRB3 antibody is a human IgG engineered for enhanced binding to FcγRIIB e.g. the “SELF” mutation (Chu et al. “Inhibition of B cell receptor-mediated activation of primary human B cells by coengagement of CD19 and FcgammaRIIb with Fc-engineered antibodies.” Mol Immunol. 2008 September; 45(15):3926-33), and/or engineered for relative enhanced binding to FcγRIIB compared to activating FcγRs e.g. V9 or V11 mutations (Mimoto et al. “Engineered antibody Fc variant with selectively enhanced FcγRIIb binding over both FcγRIIa^{R131} and FcγRIIa^{H131}”. Protein Eng Des Sel. 2013 October; 26(10): 589-598.). Such IgG variants engineered for enhanced binding to inhibitory FcγRIIB, or specifically enhanced binding affinity specifically to inhibitory FcγRIIB but not activating FcγRIIA, have been shown to increase in vivo agonist activity, and therapeutic activity, of the CD40 agonist antibody CP870,893 in animals humanized for activating and inhibitory FcγRs (Dahan et al. 2016. ‘Therapeutic Activity of Agonistic, Human Anti-CD40 Monoclonal Antibodies Requires Selective FcgammaR Engagement’, Cancer Cell, 29: 820-31).

The Fc receptor to which agonistic antibody molecule may bind in addition to LILRB3 is a receptor found on the surface of cells of myeloid origin, such as macrophages, monocytes, MDCSs, neutrophils, mast cells, basophils, or dendritic cells, or on the surface of lymphocytes such as NK cells, B cells, or certain T cells.

In other embodiments, the antibody molecules may comprise a modified Fc region having decreased binding to Fcγ receptors, such as a deglycosylated or aglycosylated variant of an IgG1 antibody molecule. Such aglycosylation may for example be achieved by an amino acid substitution of the asparagine in position 297 (N297X) in the antibody chain. The substation may be with a glutamine (N297Q), or with an alanine (N297A), or with a glycine (N297G), or with an asparagine (N297D), or by a serine (N297S). Other substitutions have e.g. been described by Jacobsen F W et al., JBC 2017, 292, 1865-1875, (see e.g. Table 1); such additional substitutions include L242C, V259C, A287C, R292C, V302C, L306C, V323C, I332C, and/or K334C.

Antibodies are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the Fv region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2.

Another part of an antibody is the Fc region (otherwise known as the fragment crystallizable domain), which comprises two of the constant domains of each of the antibody's heavy chains. As mentioned above, the Fc region is responsible for interactions between the antibody and Fc receptor.

The term antibody molecule, as used herein, encompasses full-length or full-size antibodies as well as functional fragments of full length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. A functional fragment does not always contain all six CDRs of a corresponding full-size antibody. It is appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269: 32389-93 it is described that a whole VL chain (including all three CDRs) has a high affinity for its substrate.

Molecules containing two CDR regions are described, for example, by Vaughan & Sollazzo 2001, Combinatorial Chemistry & High Throughput Screening, 4: 417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362: 367-9 and Bianchi et al., 1994, J. Mol. Biol., 236: 649-59 are referenced by Vaughan & Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362: 293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272: 30937-44, in which it is demonstrated that a range of hexapeptides derived from a CDR display antigen-binding activity and it is noted that synthetic peptides of a complete, single, CDR display strong binding activity. In Monnet et al., 1999, JBC, 274: 3789-96 it is shown that a range of 12-mer peptides and associated framework regions have antigen-binding activity and it is commented on that a CDR3-like peptide alone is capable of binding antigen. In Heap et al., 2005, J. Gen. Virol., 86: 1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. When a derivative is used it should have the same antigen binding characteristics as the corresponding full-length antibody in the sense that it binds to the same epitope on the target as the full-length antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, antibodies of human origin, humanized antibodies, chimeric antibodies, single-chain Fvs (scFv), Fab fragments, F(ab′)₂ fragments, F(ab′) fragments, disulfide-linked Fvs (sdFv), antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE, unless otherwise specified.

In some embodiments, the antibody molecule is a human antibody molecule, a humanized antibody molecule or an antibody molecule of human origin. In this context, a humanized antibody molecule means an originally non-human antibody that has been modified to increase its similarity to a human antibody. Humanized antibody molecules may, for example, originally be murine antibodies or lama antibodies. In this context, an antibody molecule of human origin means an originally human antibody molecule that has been modified.

In some embodiments, the antibody molecule is an IgG antibody.

In some embodiments, the antibody molecule is a wild-type IgG antibody.

In some embodiments, the antibody molecule is an Fc engineered IgG antibody, such as the ones mentioned above, including aglycosylated or deglycosylated IgG antibody molecules, such as those including a substitution of the asparagine in position 297, such as for example a N297Q or N297A substitution.

In some embodiments, the antibody molecule is a human IgG1 antibody. Human IgG1 antibodies correspond to murine IgG2a antibodies, so if a murine surrogate to a human IgG1 is to be used, for example for in vivo studies, a murine IgG2a format is used.

In some embodiments, the antibody molecule is a human IgG2 antibody. Human IgG2 antibodies correspond to murine IgG3 antibodies, so if a murine surrogate to a human IgG2 is to be used, for example for in vivo studies, a murine IgG3 format is used.

In some embodiments, the antibody molecule is a human IgG4 antibody. Human IgG4 antibodies correspond to murine IgG1 antibodies, so if a murine surrogate to a human IgG4 is to be used, for example for in vivo studies, a murine IgG1 format is used.

The Fc modifications may vary between human and murine antibody molecules; for example a murine N297A IgG2a antibody molecule can be used as a surrogate of a human N297Q IgG1 antibody molecule.

In some embodiments, the anti-LILRB3 antibody is a monoclonal antibody.

In some embodiments, the anti-LILRB3 antibody is a polyclonal antibody.

As outlined above, different types and forms of antibody molecules are encompassed by the invention, and would be known to the person skilled in immunology. It is well known that antibodies used for therapeutic purposes are often modified with additional components which modify the properties of the antibody molecule.

Accordingly, we include that an antibody molecule described herein or an antibody molecule used as described herein (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety. The detectable moiety allows the antibody molecule to be visualized in vitro, and/or in vivo, and/or ex vivo.

By “cytotoxic moiety”, we include a radioactive moiety, and/or enzyme, for example wherein the enzyme is a caspase, and/or toxin, for example wherein the toxin is a bacterial toxin or a venom; wherein the cytotoxic moiety is capable of inducing cell lysis.

We further include that the antibody molecule may be in an isolated form and/or purified form, and/or may be PEGylated. PEGylation is a method by which polyethylene glycol polymers are added to a molecule such as an antibody molecule or derivative to modify its behavior, for example to extend its half-life by increasing its hydrodynamic size, preventing renal clearance.

As discussed above, the CDRs of an antibody bind to the antibody target. The assignment of amino acids to each CDR described herein is in accordance with the definitions according to Kabat E A et al. 1991, In “Sequences of Proteins of Immunological Interest” Fifth Edition, NIH Publication No. 91-3242, pp xv-xvii.

As the skilled person would be aware, other methods also exist for assigning amino acids to each CDR. For example, the International ImMunoGeneTics information system (IMGT®) (http://www.imgt.org/and Lefranc and Lefranc “The Immunoglobulin FactsBook” published by Academic Press, 2001).

In some embodiments, the antibody molecule that specifically binds LILRB3 comprises one of the VH-CDR1 sequences listed in Table 1 below.

In some embodiments, the antibody molecule that specifically binds LILRB3 comprises one of the VH-CDR2 sequences listed in Table 1 below.

In some embodiments, the antibody molecule that specifically binds LILRB3 comprises one of the VH-CDR3 sequences listed in Table 1 below.

In some embodiments, the antibody molecule that specifically binds LILRB3 comprises one of the VL-CDR1 sequences listed in Table 1 below

In some embodiments, the antibody molecule that specifically binds LILRB3 comprises one of the VL-CDR2 sequences listed in Table 1 below.

In some embodiments, the antibody molecule that specifically binds LILRB3 comprises one of the VL-CDR3 sequences listed in Table 1 below.

In some embodiments, the anti-LILRB3 antibody molecule is an antibody molecule selected from the group consisting of antibody molecules wherein the three CDRs in the variable heavy chain (VH) are selected from the group consisting of:

SEQ. ID. NO: 1, SEQ. ID. NO: 2 and SEQ. ID. NO: 3;

SEQ. ID. NO: 9, SEQ. ID. NO: 10 and SEQ. ID. NO: 11;

SEQ. ID. NO: 17, SEQ. ID. NO: 18 and SEQ. ID. NO: 19; and

SEQ. ID. NO: 25, SEQ. ID. NO: 26 and SEQ. ID. NO: 27.

In some embodiments, the anti-LILRB3 antibody molecule is an antibody molecule selected from the group consisting of antibody molecules wherein the three CDRs in the variable light chain (VL) are selected from the group consisting of:

SEQ. ID. NO: 4, SEQ. ID. NO: 5 and SEQ. ID. NO: 6;

SEQ. ID. NO: 12, SEQ. ID. NO: 13 and SEQ. ID. NO: 14;

SEQ. ID. NO: 20, SEQ. ID. NO: 21 and SEQ. ID. NO: 22; and

SEQ. ID. NO: 28, SEQ. ID. NO: 29 and SEQ. ID. NO: 30.

In some embodiments, the anti-LILRB3 antibody molecule is an antibody molecule selected from the group consisting of antibody molecules comprising a VH selected from the group consisting of SEQ. ID. NOs: 7, 15, 23 and 31.

In some embodiments, the anti-LILRB3 antibody molecule is an antibody molecule selected from the group consisting of antibody molecules comprising a VL selected from the group consisting of SEQ. ID. NOs: 8, 16, 24 and 32.

In some embodiments the anti-LILRB3 antibody molecule comprises a CH having SEQ. ID. NO: 41.

In some embodiments the anti-LILRB3 antibody molecule comprises a CL having SEQ. ID. NO: 42.

TABLE 1
Specific sequences of anti-LILRB3 antibody molecules (in the
VH and VL sequences, the CDR sequences are marked in bold text)

| Antibody | | | SEQ. |
|---|---|---|---|
| clone | Region | Sequence | ID. NO: |
| A1 | VH-CDR1 | FSSYAMSWWRQAPG | 1 |
| | VH-CDR2 | SAISGSGGSTYYADSVKGR | 2 |
| | VH-CDR3 | ARRKKRERGFSGNDPVGAIDV | 3 |
| | VL-CDR1 | CTGSSSNIGAGYDVH | 4 |
| | VL-CDR2 | GNTNRPS | 5 |
| | VL-CDR3 | CSAWDDSLSGVV | 6 |
| | VH | EVQLLESGGGLVQPGGSLRLSCAASGFTFSSYAMSWVR- | 7 |
| | | QAPGKGLEWVSAISGSGGSTY- | |
| | | YADSVKGRFTISRDNSKNTLYLQMNSLRAEDTAVYYCARR | |
| | | KKRERGFSGNDPVGAIDVWGQGTLVTVSS | |
| | VL | QSVLTQPPSASGTPGQRVTISCTGSSSNI- | 8 |
| | | GAGYDVHWYQQLPGTAPKLLIYGNTNRPSGVP- | |
| | | DRFSGSKSGTSASLAISGLRSEDEADYYCSAWDDSLSGVV | |
| | | FGGGTKLTVLG | |
| A16 | VH-CDR1 | FSSYWMSWRQAPG | 9 |
| | VH-CDR2 | SRINTHGTNIDYADSVKGR | 10 |
| | VH-CDR3 | VGVAGTGWFDP | 11 |
| | VL-CDR1 | CTGSSSNIGAGYDVH | 12 |
| | VL-CDR2 | GNNNRPS | 13 |
| | VL-CDR3 | CQSYDTSLSGSV | 14 |
| | VH | EVQLLESGGGLVQPGGSLRLSCAASGFTFSSYWMSWVR- | 15 |
| | | QAPGKGLEWVSRINTHGTNIDY- | |
| | | ADSVKGRFTISRDNSKNTLYLQMNSLRAEDTAVYYCVGVA | |
| | | GTGWFDPWGQGTLVTVSS | |
| | VL | QSVLTQPPSASGTPGQRVTISCTGSSSNI- | 16 |
| | | GAGYDVHWYQQLPGTAPKLLIYGNNNRPSGVP- | |
| | | DRFSGSKSGTSASLAISGLRSEDEADYYCQSYDTSLSGSV | |
| | | FGGGTKLTVLG | |
| A20 | VH-CDR1 | FSSYSMNWVRQAPG | 17 |
| | VH-CDR2 | SAISGSGGSTYYADSVKGR | 18 |
| | VH-CDR3 | ARGLATYGLDV | 19 |
| | VL-CDR1 | CSGSSSNIGRHHVY | 20 |
| | VL-CDR2 | SNSLRPS | 21 |
| | VL-CDR3 | CAAWDDSLSGWW | 22 |
| | VH | EVQLLESGGGLVQPGGSLRLSCAASGFTFSSYSMNWVR- | 23 |
| | | QAPGKGLEWVSAISGSGGSTY- | |
| | | YADSVKGRFTISRDNSKNTLYLQMNSLRAEDTAVYYCARG | |
| | | LATYGLDVWGQGTLVTVSS | |
| | VL | QSVLTQPPSASGTPGQRVTISCSGSSSNIGRHHVY- | 24 |
| | | WYQQLPGTAPKLLIYSNSLRPSGVP- | |
| | | DRFSGSKSGTSASLAISGLRSEDEADYYCAAWDDSLSGW | |
| | | VFGGGTKLTVLG | |
| A28 | VH-CDR1 | FSSYSMNWVRQAPG | 25 |
| | VH-CDR2 | ANIKQDGTENYYVDSVEGR | 26 |
| | VH-CDR3 | ARDGDWGWGFDY | 27 |
| | VL-CDR1 | CTGSSSNIGAGYDVH | 28 |
| | VL-CDR2 | ENNKRPS | 29 |
| | VL-CDR3 | CAAWDDSLSGWW | 30 |
| | VH | EVQLLESGGGLVQPGGSLRLSCAASGFTFSSYSMNWVR- | 31 |
| | | QAPGKGLEWWANIKQDG- | |
| | | TENYYVDSVEGRFTISRDNSKNTLYLQMNSLRAEDTAVYY | |
| | | CARDGDWGWGFDYWGQGTLVTVSS | |
| | VL | QSVLTQPPSASGTPGQRVTISCTGSSSNI- | 32 |
| | | GAGYDVHWYQQLPGTAPKLLIYENNKRPSGVP- | |
| | | DRFSGSKSGTSVSLAISGLRSEDEADYYCAAWDDSLSGW | |
| | | VFGGGTKLTVLG | |

The sequences in Table 1 above are all of human origin and derived from the n-CoDeR® library, as explained in detail in Example 1.

In some embodiments, the antibody molecules that specifically bind LILRB3 described herein may also comprise one or both of the constant regions (CH and/or CL) listed in Table 2 below.

TABLE 2

| | | SEQ. |
|---|---|---|
| Region | Sequence | ID. NO: |
| CH | ASTKGPSVFPLAPSSKSTSGGTAALGCLVKDYFPEPVTVSWNSGAL- | 33 |
| | TSGVHTFPAVLQSSGLYSLSSVVTVPSSSLGTQTYICNVNHKPSNT- | |
| | KVDKKVEPKSCDKTHTCPPCPAPELLGGPSVFLFPPKPKDTLMISRTPE- | |
| | VTCVVVDVSHEDPEVKFNWYVDGVEVHNAKTKPREEQYNSTYRVVS- | |
| | VLTVLHQDWLNGKEYKCKVSNKALPAPIEKTISKAKGQPREPQVY- | |
| | TLPPSRDELTKNQVSLTCLVKGFYPSDIAVEWESNGQPEN- | |
| | NYKTTPPVLDSDGSFFLYSKLTVDKSRWQQGNVFSCSVMHEALHNHY- | |
| | TQKSLSLSPGK | |
| CL | QPKAAPSVTLFPPSSEELQANKATLVCLISDFYPGAVTVAWKADSSPV- | 34 |
| | KAGVETTTPSKQSNNKYAASSYLSLTPEQWKSHRSYSCQVTHEGSTVE | |
| | KTVAPTECS | |

The CH (SEQ. ID. NO: 33) and the (SEQ. ID. NO: 34) sequences in Table 2 above are of human origin.

As mentioned above, in some embodiments, the antibody molecules bind human LILRB3). In some such embodiments, it is preferred that the antibody molecules binds strongly to human LILRB3, i.e. that they have a low EC50 value.

In some embodiments, it is advantageous that the antibody molecule binds both to human LILRB3 and to cynomologous monkey LILRB3 (cmLILRB3 or cyno LILRB3). Cross-reactivity with LILRB3 expressed on cells in cynomologous monkey, also called crab-eating macaque or Macaca fascicularis, may be advantageous since this enables animal testing of the antibody molecule without having to use a surrogate antibody, with particular focus on tolerability.

In some embodiments, it is necessary to use a surrogate antibody to test an antibody molecule's functional activity in relevant in vivo models in mice. To ensure the comparability between the antibody molecule's effect in humans and the in vivo results for the surrogate antibody in mice, it is essential to select a functionally equivalent surrogate antibody having the same in vitro characteristics as the human antibody molecule.

In some embodiments, the antibody molecule of the present invention or used according to the invention is an antibody molecule that is capable of competing with the specific antibodies provided herein, for example capable of competing with antibody molecules comprising a VH selected from the group consisting of SEQ. ID. NOs: 7, 15, 23 and 31; and/or a VL selected from the group consisting of SEQ. ID. NOs: 8, 16, 24 and 32, for binding to LILRB3.

By “capable of competing for” we mean that the competing antibody is capable of inhibiting or otherwise interfering, at least in part, with the binding of an antibody molecule as defined herein to the specific target LILRB3.

For example, such a competing antibody molecule may be capable of inhibiting the binding of an antibody molecule described herein to LILRB3 by at least about 10%; for example at least about 20%, or at least about 30%, at least about 40%, at least about 50%, at least about 60%, at least about 70%, at least about 80%, at least about 90%, at least about 95%, or about 100%.

Competitive binding may be determined by methods well known to those skilled in the art, such as Enzyme-linked immunosorbent assay (ELISA).

ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

In some embodiments, it is of interest to use not the anti-LILRB3 antibody molecule itself but a nucleotide sequence encoding such an antibody molecule. The present invention thus encompasses nucleotide sequences encoding the above anti-LILRB3 antibody molecules.

The above described antibody molecules and nucleotide sequences, or other anti-LILRB3 antibody molecules or nucleotide sequences encoding such antibody molecules, can be used in medicine, and then such an antibody molecule and/or nucleotide sequence can be included in a pharmaceutical composition, as discussed further below.

The anti-LILRB3 antibody molecules, nucleotide sequences and/or pharmaceutical compositions can be used in the treatment of graft rejection, as discussed further below.

The anti-LILRB3 antibody molecules, nucleotide sequences and/or pharmaceutical compositions can be used in the treatment of an autoimmune disorder, as discussed further below.

The anti-LILRB3 antibody molecules, nucleotide sequences and/or pharmaceutical compositions can be used in the treatment of an inflammatory disorder, as discussed further below.

The anti-LILRB3 antibody molecules and/or nucleotide sequences can be used in the manufacture of a pharmaceutical composition for use in the treatment of graft rejection

The anti-LILRB3 antibody molecules and/or nucleotide sequences can be used in the manufacture of a pharmaceutical composition for use in the treatment of an autoimmune disorder.

The anti-LILRB3 antibody molecules and/or nucleotide sequences can be used in the manufacture of a pharmaceutical composition for use in the treatment of an inflammatory disorder.

The anti-LILRB3 antibody molecules, nucleotide sequences and/or pharmaceutical compositions can be used in treatment of graft rejection an autoimmune disorder and/or an inflammatory disorder in a patient, wherein a therapeutically effective amount of an anti-LILRB3 antibody molecule, nucleotide sequence and/or pharmaceutical composition is administered to the patient.

Examples of graft rejection that can be treated as disclosed herein include rejection in connection with an organ transplant or organ transplantation, such as transplantation of kidney, liver, heart, lungs, pancreas and intestines from a donor to a recipient, in cases where the recipient suffers from a disease or an injury that affects an organ that is replaced in the transplantation. Another example of graft rejection that can be treated as disclosed herein include rejection of an allogeneic transplant wherein stem cells, such as hematopoietic stem cells (HSCs) are collected from a matching donor and transplanted into the patient to suppress a disease and restore the patient's immune system.

At least in some embodiments, the recipient of the graft should undergo pre-conditioning by administrating agonistic LILRB3 mAb prior to transplantation. In some embodiments, the recipient of the graft should also undergo treatment with agonistic LILRB3 mAb after transplantation.

The antibody molecules, pharmaceutical compositions and treatments described herein can be used to prevent, treat or minimize rejection of the new organ or other transplant by the recipient.

Examples of autoimmune disorder, or autoimmunity, that can be treated as disclosed herein include celiac disease, diabetes mellitus type 1, sarcoidosis, systemic lupus erythematosus (SLE), Sjögren's syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), dermatomyositis (DM) and multiple sclerosis (MS).

Examples of inflammatory disorders that can be treated as disclosed herein include both chronic inflammatory disorders, such as rheumatoid arthritis (RA), systemic lupus erythematosus (SLE) and multiple sclerosis (MS), and acute inflammatory disorders, such as sepsis.

It would be known to the person skilled in medicine, that medicines can be modified with different additives, for example to change the rate in which the medicine is absorbed by the body; and can be modified in different forms, for example to allow for a particular administration route to the body.

Accordingly, we include that the antibody molecules, nucleotide sequences, plasmids and/or cells described herein may be combined with a pharmaceutically acceptable excipient, carrier, diluent, vehicle and/or adjuvant into a pharmaceutical composition. In this context, the term pharmaceutical composition can be used interchangeably with the terms pharmaceutical preparation, pharmaceutical formulation, therapeutic composition, therapeutic preparation, therapeutic formulation and therapeutic entity.

The pharmaceutical compositions described herein may comprise, or in some embodiments consist of, antibody molecules, nucleotide sequences, plasmids or cells.

The pharmaceutical compositions described herein may in some embodiments consist of or comprise plasmids comprising nucleotide sequences encoding the above described antibody molecules or comprising the above described nucleotide sequences.

The invention also comprises other therapeutic modalities, or “shapes” of drugs, such as antibody drug conjugates, fusion proteins etc, and pharmaceutical composition comprising such therapeutic modalities.

The antibody molecules, nucleotide sequences, plasmids, cells and/or pharmaceutical compositions described herein may be suitable for parenteral administration including aqueous and/or non-aqueous sterile injection solutions which may contain antioxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The antibody molecules, nucleotide sequences, plasmids, cells and/or pharmaceutical compositions described herein may be presented in unit-dose or multi-dose containers, for example sealed ampoules and vials, and may be stored in a freeze-dried (i.e. lyophilized) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the anti-LILRB3 antibody molecule will usually be from 1 mg/kg bodyweight of the patient to 20 mg/kg, or in some cases even up to 100 mg/kg administered in single or divided doses. Lower doses may be used in special circumstances, for example in combination with prolonged administration. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

Typically, a pharmaceutical composition (or medicament) described herein comprising an antibody molecule will contain the anti-LILRB3 antibody molecule at a concentration of between approximately 2 mg/ml and 150 mg/ml or between approximately 2 mg/ml and 200 mg/ml.

Generally, in humans, oral or parenteral administration of the antibody molecules, nucleotide sequences, plasmids, cells and/or pharmaceutical compositions described herein is the preferred route, being the most convenient. For veterinary use, the antibody molecules, nucleotide sequences, plasmids, cells and/or pharmaceutical compositions described herein are administered as a suitably acceptable formulation in accordance with normal veterinary practice and the veterinary surgeon will determine the dosing regimen and route of administration which will be most appropriate for a particular animal. Thus, the present invention provides a pharmaceutical formulation comprising an amount of an antibody molecule, nucleotide sequence, plasmid and/or cell of the invention effective to treat various conditions (as described above and further below). Preferably, the antibody molecules, nucleotide sequences, plasmids, cells and/or pharmaceutical compositions described herein is adapted for delivery by a route selected from the group comprising: intravenous (IV or i.v.); intramuscular (IM or i.m.) or subcutaneous (SC or s.c.).

The present invention also includes antibody molecules, nucleotide sequences, plasmids, cells and/or pharmaceutical compositions described herein comprising pharmaceutically acceptable acid or base addition salts of the target binding molecules or parts of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms of the agents according to the present invention. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The antibody molecules, nucleotide sequences, plasmids and/or cells described herein may be lyophilized for storage and reconstituted in a suitable carrier prior to use. Any suitable lyophilization method (e.g. spray drying, cake drying) and/or reconstitution techniques can be employed. It will be appreciated by those skilled in the art that lyophilization and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilized (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilization) when re-hydrated.

The anti-LILRB3 antibody molecules, nucleotide sequences and pharmaceutical compositions described herein can be used use in the treatment of graft rejection or autoimmunity in a subject or patient. Herein, the terms subject and patient are used interchangeably

“Patient” (or subject) as the term is used herein refers to an animal, including human, that has been diagnosed as suffering from graft rejection or autoimmunity and/or that exhibits symptoms of suffering from graft rejection or autoimmunity.

In some embodiments, the patient (or subject) is an animal, including human, that has been diagnosed as suffering from graft rejection or autoimmunity. In some embodiments, the patient (or subject) is an animal, including human, that will undergo a transplantation and therefore being in the risk of graft rejection; the treatment discussed herein is then a carried out as a preventive treatment or for preventive purposes.

In some embodiments, the patient (or subject) is a mammalian or non-mammalian animal, including human, that has been diagnosed as having graft rejection or autoimmunity and/or that exhibits symptoms of graft rejection or autoimmunity.

The treatments may be administered as a course of treatment, which is to say that the therapeutic agent is administered over a period of time. The length of time of the course of treatment will depend on a number of factors, which could include the type of therapeutic agent being administered, the type of disease or condition being treated, the severity of the disease or condition being treated, and the age and health of the patient, amongst others reasons.

By “during the treatment”, we include that the patient is currently receiving a course of treatment, and/or receiving a therapeutic agent, and/or receiving a course of a therapeutic agent.

### BRIEF DESCRIPTION OF THE DRAWINGS

In the examples below, reference is made to the following figures:

FIG. 1. Generation of fully human mAb against human LILRB3. FIG. 1A: Screening of generated LILRB3 clones. FMAT was performed and scFv clones screened against LILRB3 target and LILRB1 non-target-transfected cells. MFI was calculated, with target-specific scFvs depicted in lighter color and non-target scFvs in darker color. FIG. 1 B: Screening of LILRB3 mAb by flow cytometry. Peripheral blood mononuclear cells (PBMCs) or LILR-transfected CHO-S cells were incubated with His-tagged scFv supernatants, followed by anti-His-AF647 staining. Where transfected CHO-S cells were used, LILRB1- and LILRB2-transfected CHO-S cells were used as non-targets for LILRB3. Antibody clones were compared against both gated monocytes and target transfected CHO-S cells using the TIBCO Spotfire software, with LILRB3 specific clones highlighted in light grey, non-specific clones in dark grey, and the irrelevant isotype control in grey. FIG. 1 C: Specificity of LILRB3 clones against human LILR-transfected 2B4 cells. LILRB3 mAb were tested against cells transfected with the indicated LILR family members by flow cytometry; a representative clone (A16) is presented. FIGS. 1 D and E: Testing the specificity of LILRB clones against primary cells by flow cytometry. PBMCs (FIG. 1 D) or whole blood (FIG. 1 E) stained with either APC-labelled LILRB3 (clone A16) or hIgG1 isotype as well as various leukocyte surface markers, as indicated. Histograms are representative plots of multiple donors: monocytes and B cells (n=12), T cells and NK cells (n=12) and neutrophils (n=6).

FIG. 2. Characterization of LILRB3 antibodies. FIG. 2A: LILRB3 mAb affinity assessed by SPR. LILRB3-hFc recombinant protein was immobilized and various LILRB3 mAb flowed across the chip. KD values were calculated using the Biacore™ T100 Evaluation Software. Representative LILRB3 clones are shown. FIG. 2 B: Ability of generated mAb to cross-block binding of LILRB3 mAb (a commercial LILRB3 mAb; (clone 222821, R&D Systems, UK)). PBMCs were stained with unconjugated LILRB3 antibody clones and subsequently stained with a directly-conjugated commercial LILRB3 mAb and analyzed by flow cytometry; representative clones displayed, as indicated. The isotype control (iso ctrl) is shaded in grey, clone 222821 alone in black and in combination with indicated LILRB3 clones in grey line. FIG. 2 C: LILRB3 domain epitope mapping. HEK293F cell transfected with either WT LILRB3 (full-length extracellular portion), LILRB3-D1-3, LILRB3-D1-2 or LILRB3-D1 were stained with LILRB3 clones, followed by an anti-hIgG-PE secondary antibody staining. Schematic of domain constructs generated and restriction digest of each construct shown. Histograms showing staining of two representative clones differentially binding to WT (D4), D3, D2 and D1-expressing cells, as indicated (n=3 independent experiments). FIG. 2 D: LILRB3 2B4 reporter cells were treated with 10 μg/ml LILRB3 antibodies overnight to assess agonism or antagonism. GFP expression was then measured by flow cytometry; representative clones shown.

FIG. 3. LILRB3 ligation regulates T cell activation and proliferation. CFSE-labelled PBMCs were stimulated with antibodies against human CD3 and CD28 in the presence or absence of isotype control (iso ctrl) or LILRB3 mAb (10 μg/ml) and proliferation measured through CFSE dilution after 3-5 days. FIG. 3A: Assessing T cell activation and proliferation following treatment. Light microscopy images following PBMC stimulation in culture. CD8+ T cell proliferation was assessed through CFSE dilution; representative histograms shown. FIG. 3 B: LILRB3 mAb were deglycosylated (Degly) through PNGase-treatment, as confirmed by SDS-PAGE; representative clones shown. FIG. 3 C: Assessing the effects of deglycosylated LILRB3 mAb on T cell proliferation. CFSE dilution of CD8+ T cells, treated with various LILRB3 mAb was assessed by flow cytometry. Data normalized to anti-CD3/CD28-treated samples and mean represented by solid line; representative clones shown. Two-tailed paired T-test performed and stars represent level of significant difference compared to iso ctrl (*** p<0.005); n=13-20 independent donors (each dot represents an individual donor).

FIG. 4. LILRB3 ligation modulates macrophage phagocytosis. FIG. 4A: Human MDMs were stained with anti-CD14 and anti-LILRB3 (A16) and analyzed by flow cytometry. FIG. 4 B: MDMs were treated with deglycosylated isotype control (iso ctrl) or LILRB3 mAb (10 μg/ml) prior to co-culture with CFSE⁺ rituximab-opsonized target cells; and phagocytosis was defined as the number of gated live cells that were double positive (CD16⁺ CFSE⁺ cells), as a percentage of total MDMs (CD16⁺ cells), using the following equation:

(Double positive MDM/Total MDM)×100=% positive MDMs

FIG. 4 C: The effect of deglycosylated LILRB3 mAb on phagocytosis. Each donor was performed in triplicate and the mean is represented by a solid line (n=4-6 healthy donors); representative clones shown. Two-tailed paired T-test was performed and stars represent level of significant difference compared to isotype control (* p<0.05, *** p<0.0005). FIG. 4 D: The effect of deglycosylated LILRB3 mAb on phagocytosis assessed by confocal microscopy. LILRB3-treated MDMs (grey) were co-cultured with CFSE-labelled B cells (light grey), fixed in 4% PFA and membrane glycoproteins stained with biotinylated WGA. Cells were then incubated with a secondary streptavidin-conjugated AF635 and analyzed by confocal microscopy.

FIG. 5. LILRB3 ligation induce induces tolerance in vivo. Fully reconstituted humanized mice (≥50% circulating hCD45+ leukocytes) were generated and the expression of human LILRB3 was confirmed on CD14+ myeloid cells. FIG. 5A: Representative flow cytometry histogram showing LILRB3 expression on hCD45+ bone marrow hCD14⁺ myeloid cells; isotype control in solid dark grey, LILRB3 in solid light grey. FIG. 5 B: Humanized mice were injected with 200 μg LILRB3 mAb (clone A1) or an isotype-matched (hIgG1) control mAb on day 0 and 4, i.v. and intraperitoneal (i.p.), respectively. On day 7, mice were injected i.p. with 1×10⁷ non-autologous luciferase⁺ human lymphoma cells. Lymphoma cell growth was monitored over time using an IVIS imager, representative images from 3 independent experiments shown (n=3 mice/group).

FIG. 6. Human LILRB3 ligation reprograms human primary myeloid cells. Freshly isolated human peripheral CD14⁺ monocytes were treated with an isotype control (iso ctrl) or a human LILRB3 mAb (clone A1). FIG. 6A: Monocyte morphology following treatment. Light microscopy images following overnight treatment of freshly-isolated CD14⁺ monocytes with indicated mAb in culture. FIG. 6 B: Transcriptomic analysis of LILRB3-treated monocytes. RNA was extracted from cells following mAb treatment (˜18 hours) and subjected to RNA sequencing. The left panel depicts a list of genes that were significantly upregulated and the right panel depicts genes that were significantly downregulated compared to iso ctrl treated-cells (n=4; each row represents an individual donor). FIG. 6 C: Ligation of LILRB3 on primary human CD14⁺ monocytes induces M2-polarized genes. GSEA graphs showing a significant enrichment for M2-polarizing genes in LILRB3-treated monocytes versus isotype control, respectively. UP; upregulated, NES; normalized enrichment score=−1.68; FWER; familywise-error rate p<0.001. FIG. 6 D: qPCR analysis of selected genes following LILRB3 ligation on monocytes. Data were normalized to GAPDH mRNA levels and standardized to the levels of isotype control-treated monocytes. Fold difference data were log 10 transformed. One-way ANOVA with Bonferroni's multiple comparisons test was performed, n=3 independent donors (** p<0.005, *** p<0.0005). FIG. 6 E: GSEA analysis showing negative correlation with ‘IFN-γ’ (NES=−2.17; FWER p<0.001), ‘IFN-α’ (NES=−2.3; FWER p<0.001) and ‘allograft rejection’ (NES=−1.58; FWER p=0.14) signaling elements and positive correlation with ‘oxidative phosphorylation’ (NES=2; FWER p<0.001). FIG. 6 F: Schematic diagram demonstrating the immunosuppressive function of LILRB3 following ligation on APCs.

### EXAMPLES

Specific, non-limiting examples which embodies certain aspects of the invention will now be described.

### Materials and Methods

### Ethics Statement

All research with human samples and mice was performed in compliance with institutional guidelines, the Declaration of Helsinki and the US Department of Health and Human Services Guide for the Care and Use of Laboratory Animals. The Committee on Animal Care at Massachusetts Institute of Technology (MIT) reviewed and approved the studies described here. All human samples (adult peripheral blood and fetal liver) were collected anonymously with informed consent by a third party and purchased for research. For human peripheral blood, ethical approval for the use of clinical samples was obtained by the Southampton University Hospitals NHS Trust; from the Southampton and South West Hampshire Research Ethics Committee following provision of informed consent. Primary chronic lymphocytic leukemia (CLL) samples were released from the Human Tissue Authority licensed University of Southampton, Cancer Science Unit Tissue Bank as part of the LPD study LREC number 228/02/T.

### Hematopoietic Stem/Progenitor Cells (HSPCs) Isolation and Generation of Humanized Mice

Human fetal livers were obtained from aborted fetuses at 15-23 weeks of gestation, in accordance with the institutional ethical guidelines (Advanced Bioscience Resources, Inc., Calif., USA). All women gave written informed consent for the donation of their fetal tissue for research. Fetuses were collected within 2 hours of the termination of pregnancy. Fetal liver tissue was initially cut into small pieces and digested with collagenase VI (2 mg/ml in Dulbecco's modified Eagle's medium [DMEM]) for 30 minutes at 37° C. with periodic mixing. Single-cell suspensions were prepared by passing the digested tissue through a 100 μm cell strainer (BD Biosciences, NJ, USA). CD34⁺ cells were purified with the use of a CD34⁺ selection kit (Stem Cell Technologies, Vancouver, BC, Canada); the purity of CD34⁺ cells was 90%-99%. Viable cells were counted by trypan blue exclusion of dead cells. All cells were isolated under sterile conditions.

NSG mice were purchased from the Jackson Laboratories (Bar Harbor, Me., USA) and maintained under specific pathogen-free conditions in the animal facilities at MIT. To reconstitute mice, newborn pups (less than 2 days old) were irradiated with 100 cGyusing a Gamma radiation source injected intracardially with CD34+CD133+ cells (approximately 2×10⁵ cells/recipient), as reported previously (25). Around 12 weeks of age, human leukocyte reconstitution was determined by flow cytometry of peripheral blood mononuclear cells (PBMCs). Chimerism, or the level of human leukocyte reconstitution, was calculated as follows: % CD45+ human cell/(% CD45+ human cell+% CD45+ mouse cell). Mice with 40% human CD45+ leukocytes were used in the study.

### Cell Culture

Cell lines were grown at 37° C. in either RPMI 1640 medium supplemented with 10% fetal calf serum (FCS) (Sigma-Aldrich, UK), 100 U/ml Penicillin-Streptomycin, 2 mM glutamine and 1 mM pyruvate (Thermo Fisher Scientific, UK) in a humidified incubator with 5% CO₂, Freestyle 293F media, in 8% CO₂, shaking at 130 rpm, or Freestyle CHO media (Thermo Fisher Scientific, UK) with 8 mM glutamine, in 8% CO₂, shaking at 140 rpm.

### Antibody Generation and Production

### Generation of LILRB3 Antibodies.

Selection of various LILRB3-specific mAb was performed using the n-CoDeR® phage display library (26). Three consecutive panning rounds were performed, as well as a pre-panning step. In the panning, Fc fusion proteins containing the extracellular domains of LILRB1, LILRB2 or LILRB3 (LILRB-Fc) were used as non-targets or targets, respectively. These proteins were produced in transiently transfected HEK293 cells followed by purification on protein A, as described previously (27). CHO-S cells transiently transfected to express the various LILRB proteins were also used as targets/non-targets in the panning.

In panning 1, BioInvent n-CoDeR® scFv were selected using biotinylated in-house produced recombinant LILRB-human (h) Fc recombinant fusion proteins (captured with streptavidin-coated Dynabeads®) with or without competition or LILRB-hFc coated to etched polystyrene balls (Polysciences, US)/plastic immunotubes. Binding phages were eluted by trypsin digestion and amplified on plates using standard procedures (28). The amplified phages from panning 1 were used for panning 2, the process repeated, and the amplified phages from panning 2 used in panning 3. In the third panning round however, amplified phages from all 3 strategies were combined and selected against LILRB transiently transfected CHO-S cells.

Next, the LILRB3-positive scFv cassettes from the enriched phage repertoires from panning 3 were re-cloned to allow soluble scFv expression in E. coli. The soluble scFv fragments expressed by individual clones were tested for binding against LILRB-transfected CHO-S cells using Flourometric Microvolume Assay Technology (FMAT), and recombinant LILRB protein by Enzyme-linked immunosorbent assay (ELISA). This allowed the identification of clones binding specifically to LILRB3. Clones were then further reduced in a tertiary screen against CHO-S cells expressing LILRB1-3 and primary cells (PBMCs). Clones showing specific patterns of binding to a single LILRB were sequenced, yielding LILRB1-3-specific mAb.

### Production of Full-Length IgG's.

The unique scFv identified above were cloned into a eukaryotic expression system allowing transient expression of full-length IgG in HEK293-EBNA cells. The antibodies were then purified from the culture supernatants using Protein A-based affinity chromatography as previously described (29).

### Production of Deglycosylated IgG.

To allow dissection of Fc- and Fab-dependent effector functions, IgG were deglycosylated using PNGase F (Promega) with 0.05 U of PNGase/μg of IgG, at 37° C. for at least 15 hours. Deglycosylation was confirmed by reduction in size of the heavy chain on SDS-PAGE.

### Production of Domain Mutant Constructs

Using wild-type LILRB3 cDNA isolated from a healthy donor PBMCs, a series of domain mutant DNA constructs were generated by overlap PCR to express 1, 2 or 3 LILRB3 Ig-like domains (with domains identified based on annotations in Uniprot). The gene constructs were then cloned into pcDNA3.

### Cell Transfections

10×10⁶ HEK293F cells were transiently transfected with 10 μg of plasmid DNA by lipofection using 233 fectin with Optimem 1 Media (Thermo Fisher Scientific, UK).

### Preparation of Human Leukocytes and Generation of Monocyte-Derived Macrophages (MDMs)

Whole blood was acquired with informed consent from healthy volunteers. PBMC were isolated from leukocyte blood cones (Blood Transfusion Services, Southampton General Hospital). Isolation was performed by gradient density centrifugation using lymphoprep (Axis Shield, UK). MDMs were generated from healthy peripheral blood human monocytes as before (30). Briefly, PBMCs were plated at 2×10⁷ cells/well in a 6-well plate (Corning, UK) with 1% human AB serum (Sigma-Aldrich, UK) and incubated at 37° C. for 2 hours. Non-adherent cells were washed away and the adherent monocytes (>90% CD14⁺) were incubated at 37° C. overnight with 5% CO₂. The following day 100 ng/ml human recombinant M-CSF (in house) was added to each well. Media and cytokine were replenished twice during culture and cells were then harvested on day 7-8.

### Macrophage Phagocytosis Assay

Human MDMs generated as described above, were plated at 1×10⁵ cells/well in a 96-well flat-bottom plate. MDMs were treated with 10 μg/ml LILRB3 antibodies for 2 hours and washed. Primary chronic lymphocytic leukemia (CLL) cells, labelled with 5 μM CFSE (Sigma-Aldrich, UK), were opsonized with rituximab for 25 minutes at 4° C. (or herceptin as an isotype control). MDMs and target CLL cells were then co-cultured for 1 hour at 37° C., at a 1:5 ratio, respectively, before staining with 10 μg/ml CD16-APC (BioLegend, UK) for 15 minutes at room temperature in the dark. Cells were washed, harvested, analyzed by flow cytometry and % phagocytosis calculated as follows: (% double-positive MDM)/(% total MDM)×100.

### Flow Cytometry

For cell surface staining of PBMCs or whole blood, cells were blocked with 2% human AB serum (Sigma-Aldrich, UK) for 10 minutes on ice and then stained with the relevant APC-labelled mAb or hIgG1 isotype (BioInvent, Sweden), alongside the following cell surface markers: CD14-PE (eBioscience, UK), CD20-A488 (fluorescent labelled rituximab, in house), CD3-PE-Cy7, CD56-APC-Cy7 or CD15-Pacific Blue and CD66B-FITC mAb (all BioLegend, UK). Cells were stained for 30 minutes at 4° C. and then were washed twice, first in 10% red blood cell (RBC) lysis buffer (Serotec, UK) and then FACS wash (PBS, 1% BSA, 10 mM NaN3), before acquisition on a FACSCalibur or FACSCanto II (BD Biosciences, USA) and analyzed with FCS Express V3 (De Novo Software).

For assays to determine if mAb bound to similar cross-blocking epitopes 1×10⁶ PBMCs were blocked with 2% human AB serum for 10 minutes and stained with 10 μg/ml unconjugated LILRB3 mAb for 30 minutes at 4° C. The cells were then stained with directly-conjugated commercial LILRB3 mAb (clone 222821; R&D Systems, UK) for 20 minutes at 4° C., before washing and acquisition using a FACSCalibur.

For LILRB3 epitope mapping studies, LILRB3-domain mutant-transfected HEK293F cells were stained with the relevant LILRB3 mAb for 25 minutes at 4° C., washed twice, stained with an anti-human-PE secondary (Jackson ImmunoResearch, USA) for 20 minutes at 4° C., before washing and acquisition using a FACSCalibur.

For staining of 2B4 reporter cells expressing LILR-A1, -A2, -A5, -B1, -B2, -B3, -B4, or -B5 (or non-transfected controls) cells were stained with 10 μg/ml LILRB mAb and incubated at 37° C. with 5% CO₂, overnight. The following day, the cells were washed and stained with a secondary anti-hIgG antibody (Jackson ImmunoResearch, USA) at 4° C., for 45 minutes. The cells were washed and acquisition performed using a FACScan (BD Biosciences, USA) and analysis using Cell Quest (BD Biosciences, USA).

Flow cytometry data were analyzed with FCS Express V3 (De Novo Software) and FlowJo.

### Surface Plasmon Resonance (SPR)

SPR was performed with the Biacore T100 (GE Healthcare, UK) as per the manufacturer's instructions. LILRB3-hFc recombinant protein (the extracellular LILRB3 domain with a human Fc tag) was used as the ligand and immobilized by amine coupling onto a series S sensor chip (CM5). Various LILRB3 mAb were used as “analytes” and flowed across the chip, and SPR measured. KD values were calculated from the ‘Univalent’ model of 1:1 binding by Kd [1/s]/Ka [1/Ms], using the Biacore™ T100 Evaluation Software (GE Healthcare, UK).

### T Cell Proliferation Assay

PBMCs (1-2×10⁷) were labelled with 2 μM CSFE at room temperature for 10 minutes. Cells were subsequently resuspended in serum-free CTL medium (Immunospot, Germany) and plated at 1×10⁵ cells/well in a 96-well round-bottom plate (Corning, UK). Cells were then stimulated with 0.02 μg/ml CD3 (clone OKT3, University of Southampton), 5 μg/ml CD28 (clone CD28.2; BioLegend, UK) and 10 μg/ml LILRB3 antibodies or a relevant isotype. Plates were then incubated at 37° C. for 4 days, after which time cells were stained with 5 μg/ml CD8-APC (clone SK1; BioLegend, UK), harvested and CSFE dilution measured by flow cytometry, as a readout for T cell proliferation.

### In Vivo Allograft Assay

Fully reconstituted humanized mice (≥40% circulating hCD45+ leukocytes) were injected with 200 μg LILRB3 mAb (clone A1) or an isotype-matched (hIgG1) control on day 0 and day 4, i.v. and i.p, respectively. On day 7 cohorts of mice were injected i.p. with 1×10⁷ luciferase-positive human ‘double-hit’ B cell lymphomas (25, 31), derived from unrelated unmatched donors. Lymphoma cell growth was monitored over time using an IVIS Spectrum-bioluminescent imaging system, as before (25).

### Transcriptome Analysis

To assess LILRB3-mediated transcriptional changes on monocytes, human peripheral blood monocytes were isolated from freshly prepared PBMCs taken from healthy donors using an EasySep™ Human Monocyte Enrichment Kit (negative selection cell; Stem-Cell Technologies, USA). Cells were incubated in CTL medium supplemented with 100 U/ml Penicillin-Streptomycin, 2 mM Glutamine and HEPES buffer and treated with 10 μg/ml of an isotype control or an agonistic LILRB3 mAb (clone A1; hIgG1). 18 hours later cells were lysed in RLT lysis buffer containing β-mercaptoethanol and total RNA extracted using the RNeasy micro kit (Qiagen, USA). Total RNA was assessed for quality and quantified using a total RNA 6000 Nano LabChip on a 2100 Bioanalyzer (Agilent Inc., USA) and cDNA libraries prepared and sequenced according to the Illumina TruSeq RNA Sample Preparation Guide for SMARTer Universal Low Input RNA Kit (Clontech, USA) and a HiSeq 2000 system (Illumina, USA). RNAseq outputs were aligned to hg19, using Bowtie2 v2.2.3 (32). The number of mapped reads were quantified by RSEM v1.2.15 (33). Differential expression analysis between paired samples before and after treatment was performed using edgeR (34) with p<0.05 and >2 fold-change cut-offs. Differentially expressed genes were annotated using online functional enrichment analysis tool DAVID (http://david.ncifcrf.gov/) (35). Gene set enrichment analysis (GSEA) was performed using Broad Institute Software (36), with the gene list pre-ranked according to log FC values from the edgeR output. For comparison of gene-set expression, M1 and M2 macrophage gene sets (37) were obtained from the Molecular Signature Database (http://software.broadinstitute.org/gsea/msigdb/). Heatmaps were visualized with MeV (38).

Quantitative PCR (qPCR)

Probe-based qPCR was used to amplify cDNA in 20 μl reactions performed in triplicate for each sample condition in a 96-well PCR plate (Bio-Rad, UK). Each reaction comprised of 48 ng cDNA, 10 μl platinum qPCR mix (Life Technologies, UK), 8 μl DEPC water and 1 μl of gene-specific 20× PrimeTime probe/primer mix, as per manufacturer's protocol. The 96-well plate containing the PCR reagents were run in a C1000 Thermal Cycler CFX96 Real-time System PCR machine (Bio-Rad, Kidlington, UK). The CFX manager software (Bio-Rad, Kidlington, UK), was used for data acquisition and analysis of gene expression initially recorded as cycle threshold values (Ct). The Ct values were normalized to housekeeping gene Glyceraldehyde 3-phosphate dehydrogenase (GAPDH) and standardized to gene expression levels in isotype control-treated cells.

### Statistics

Paired two-tailed T-tests were performed for both the phagocytosis and T cell proliferation data; straight bars indicate median values. On bar graphs, where at least 3 experiments were performed, error bars represent standard deviation. One-way ANOVA with Bonferroni's multiple comparisons test was performed for qPCR data analysis. Statistical analysis was performed using GraphPadPrism (v5 or 6).

### Results

### Generation of a Panel of Specific LILRB3 mAb

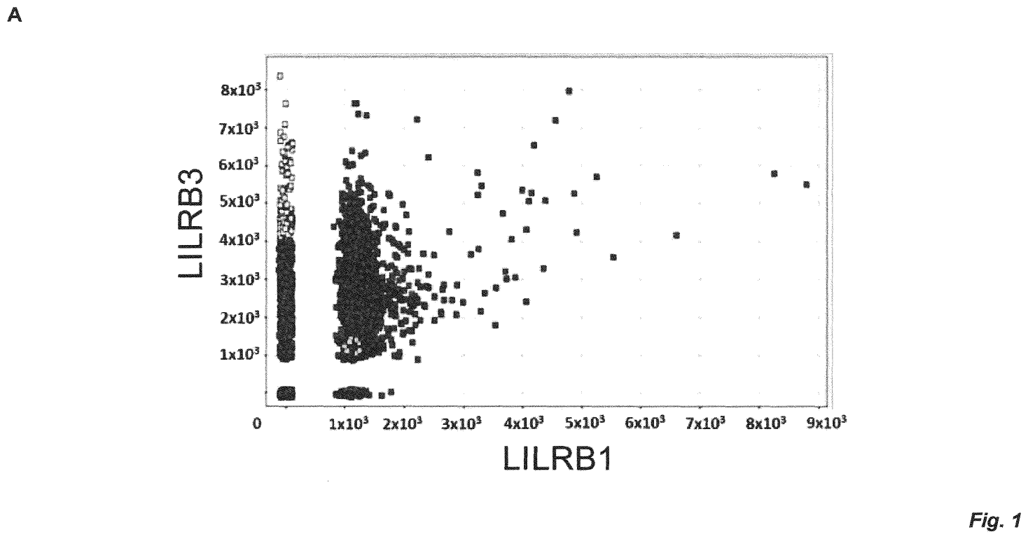

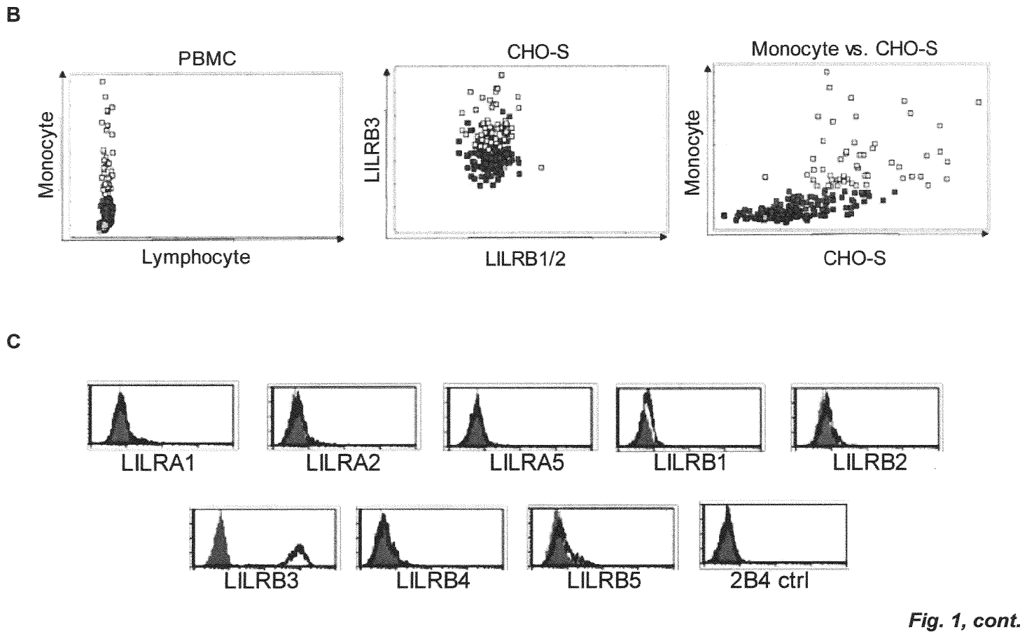

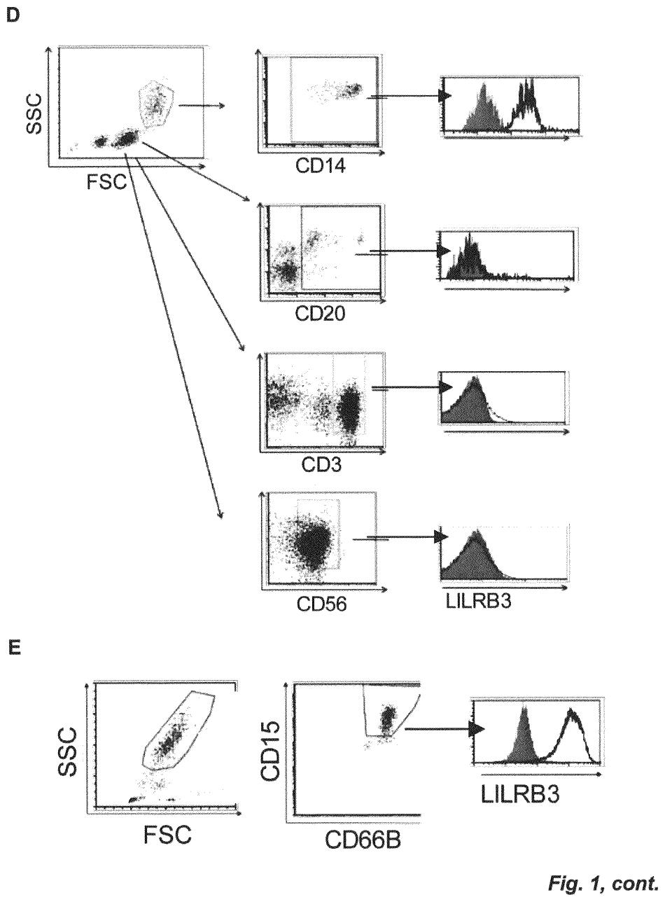

To study the protein expression and function of human LILRB3, antibodies against LILRB3 were identified using a human phage display library. Selection was performed using target LILRB3 protein (in solution, coated to a plastic surface or expressed on cells) and by selecting against homologous non-target LILRB1 and LILRB2 proteins. After three rounds of phage panning and enrichment, successful selection of clones specific for LILRB3 was confirmed by flow cytometry and ELISA (data not shown). Subsequently, target-specific phage-bound scFv clones were converted to soluble scFv and screened by FMAT and ELISA. Fluorescent intensity for each clone was plotted and target versus non-target specificity displayed (FIG. 1A). Successful clones were selected based on binding LILRB3 and lack of cross-reactivity to LILRB1 and LILRB2. Selected clones were then sequenced and tested for binding against primary cells and transfectants (FIG. 1B). Once the target-specific clones were chosen and converted to IgG, specificity was reconfirmed by screening against a panel of LILR-expressing 2B4 reporter cell lines (FIG. 10). In total 16 LILRB3-specific antibodies were identified for further study. Staining of PBMCs or whole blood with these LILRB3 mAb showed predominant staining of monocytes (FIG. 1D) and neutrophils (FIG. 1E), in agreement with previous reports (39). LILRB3-specific clones were further tested and confirmed to have no cross reactivity to the mouse orthologue, PIR-B (data not shown).

LILRB3 mAb Bind with High Affinity and Map to Different Epitopes

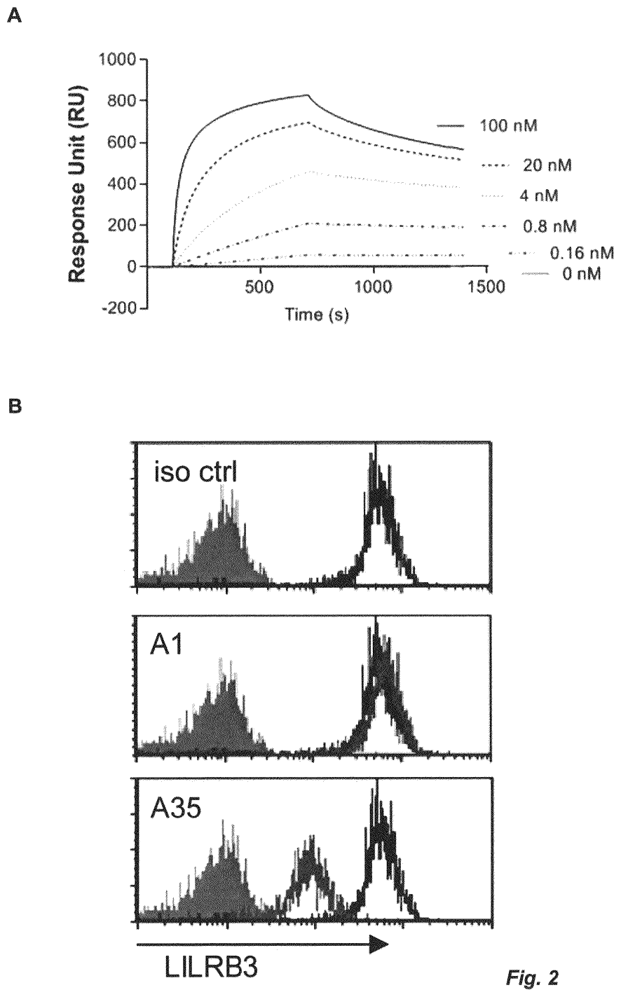

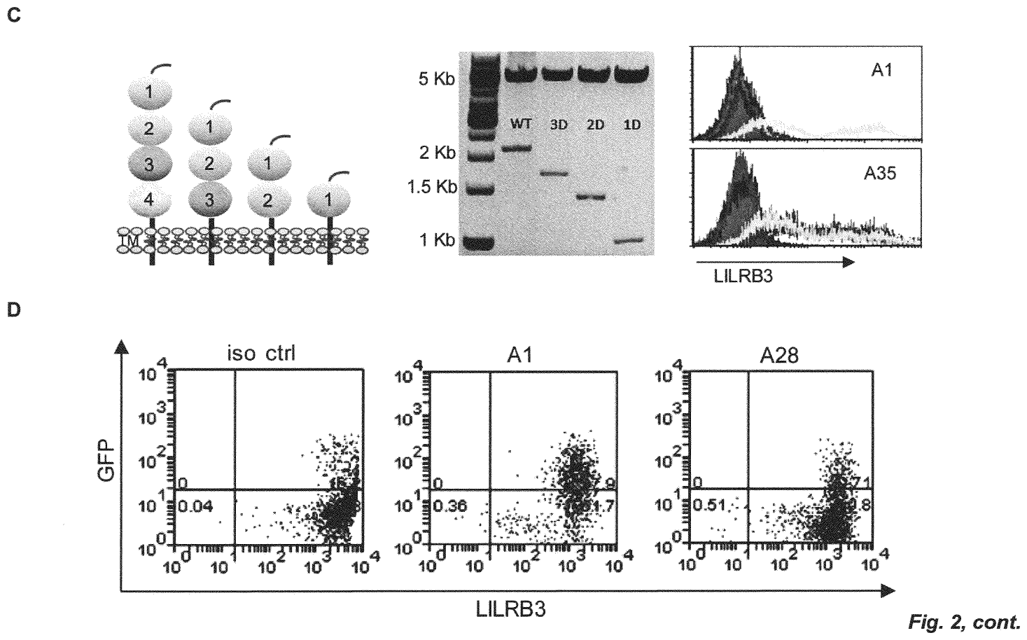

The LILRB3-specific mAb were then tested for their binding properties. SPR analysis showed that all LILRB3 clones bound to recombinant LILRB3-hFc protein in a dose-dependent manner (FIG. 2A) and displayed a range of affinities, represented by A16 (8.16×10⁻¹⁰). Interestingly, all of the mAb had similar association rates (˜105), but varied in their dissociation rates by three orders of magnitude (˜10⁻³-10⁻⁶).

Epitope mapping studies were then performed. Some mAb were able to block the binding of a commercial LILRB3 mAb (e.g., A35), suggesting a shared or proximally-related epitope; whilst others could not (e.g., A1), indicating binding elsewhere (FIG. 2B). Binding specificities were further confirmed with a series of LILRB3 domain (FIG. 2 D) mutants displaying either all four extracellular domains (WT), three, two or one domain, transiently transfected into HEK293F cells. Binding to these cells showed two groups of mAb: those that bound to the WT, D3 and D2 expressing cells; and those that bound only the WT-transfected cells (FIG. 2B), indicating binding within D4 (exemplified by A1), respectively (FIG. 2C). These data demonstrate that highly specific, fully human IgG1 mAb were raised against LILRB3. Epitope mapping revealed that, although conserved residues seem to be present in all 4 domains, LILRB3 mAb bind to either of the two distinct extracellular dominant epitopes, located within D2 and D4, respectively.

Furthermore, reporter cells transfected with the extracellular domain of LILRB3, fused with the human CD3 cytoplasmic domain were used to investigate whether the generated mAb were able to crosslink the receptor. Signaling through these hybrid cells results in the expression of GFP under the NFAT promoter (40). We were able to identify two distinct groups of LILRB3 mAb, those capable of inducing signaling (e.g. A1) and those being inert (e.g. A28) upon binding to the receptor (FIG. 2D).

### LILRB3 Ligation Modulates T Cell Activation and Proliferation

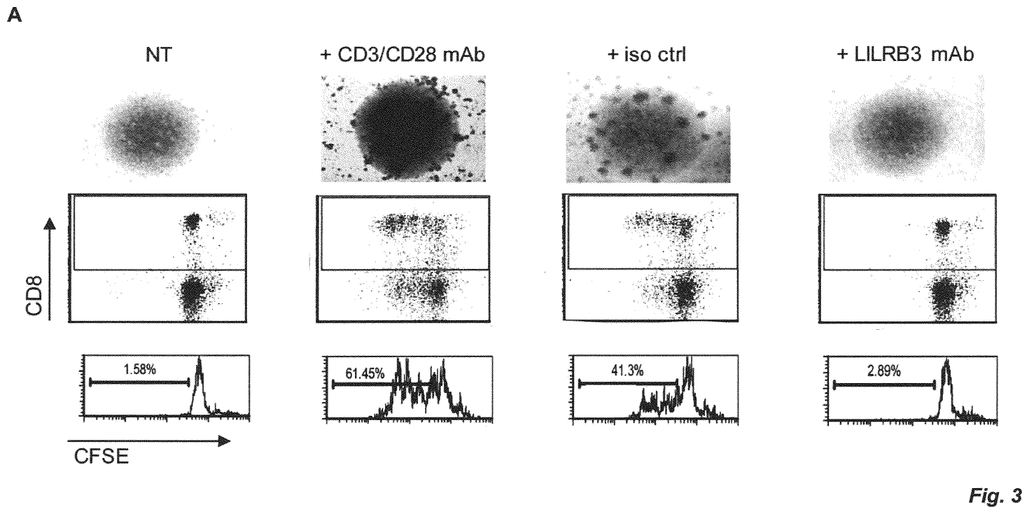

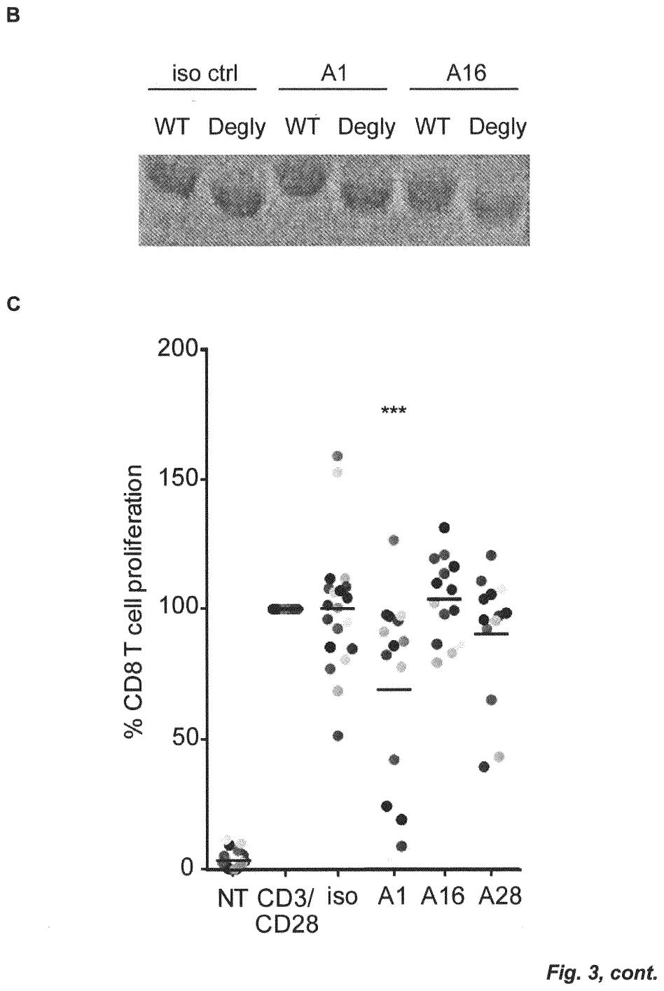

Next, we sought to investigate the effect of these mAb on cellular effector functions. LILRB1 has previously been shown to inhibit T cell responses; either by causing dephosphorylation in the CD3 signaling cascade, or competing with CD8 for HLA-I binding (41, 42). LILRBs have also been shown to inhibit T cell responses indirectly by rendering antigen-presenting cells (APC), such as monocytes and DCs tolerogenic, through the induction of CD8+ T suppressor cells (10, 12, 43). In order to investigate the immunomodulatory potential of LILRB3 and its ability to regulate adaptive immune responses, we tested LILRB3 mAb in PBMC assays, measuring T cell proliferation in response to anti-CD3/CD28 stimulation. T cell activation and proliferation was successfully driven by CD3 and CD28 antibodies, demonstrated by cell clustering and CFSE dilution (FIG. 3A).

Fcγ receptors (FcγRs) are known to mediate the effects of human IgG (29, 44-46), therefore, to study the direct F(ab):receptor-mediated effects of the LILRB3 mAb on T cell proliferation, they were first deglycosylated to eliminate effects mediated by FcγR-IgG interactions (47). SDS-PAGE confirmed a decrease in molecular weight of deglycosylated mAb (Degly) compared to wild-type (WT) controls, indicative of successful deglycosylation (FIG. 3 B). The mAb were then introduced to the T cell proliferation assay detailed above. Successful T cell proliferation driven by CD3 and CD28 antibodies was assessed in 20 different donors, showing a significant increase in CD8+ T cell proliferation, compared to controls (p<0.0001) (FIG. 3 C). The majority of the LILRB3 mAb significantly inhibited CD8+ T cell proliferation, represented by clone A1, when compared to the human IgG1 isotype control (p=0.0001; FIG. 3 C). A28 also exhibited a trend for inhibited proliferation, but A16 appeared to have no inhibitory effect. These data demonstrate that targeting LILRB3 can modulate T cell responses in either direction in a clear mAb-specific manner, with some delivering LILR3B-agonistic properties (enhanced inhibition) such as A1. When the assay was repeated with isolated T cells in the same manner, no inhibition was seen confirming that the APCs within the PBMCs, most likely monocytes, were responsible for the effects observed (data not shown). This result was expected, given the lack of expression of LILR3B on T cells.

### LILRB3 mAb Modulate Macrophage Effector Function

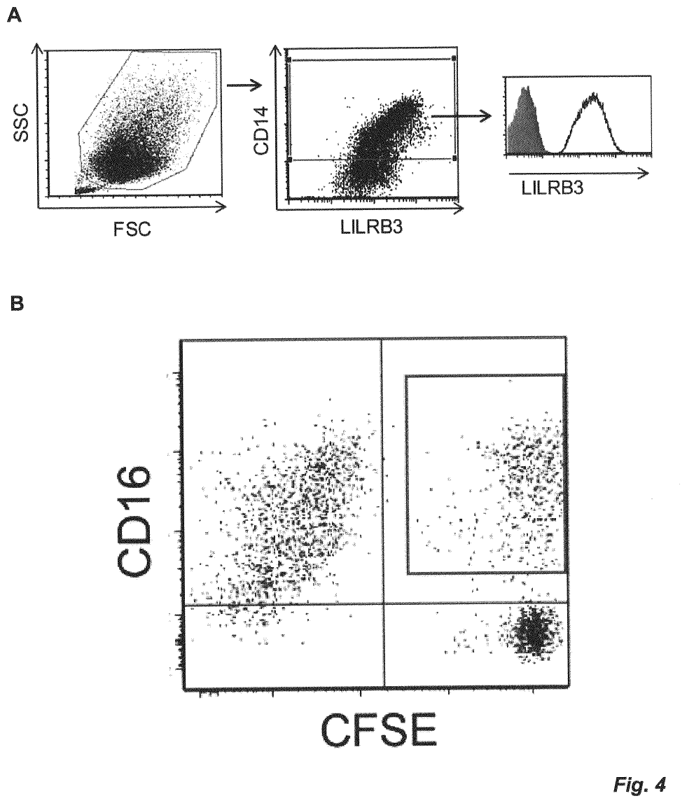

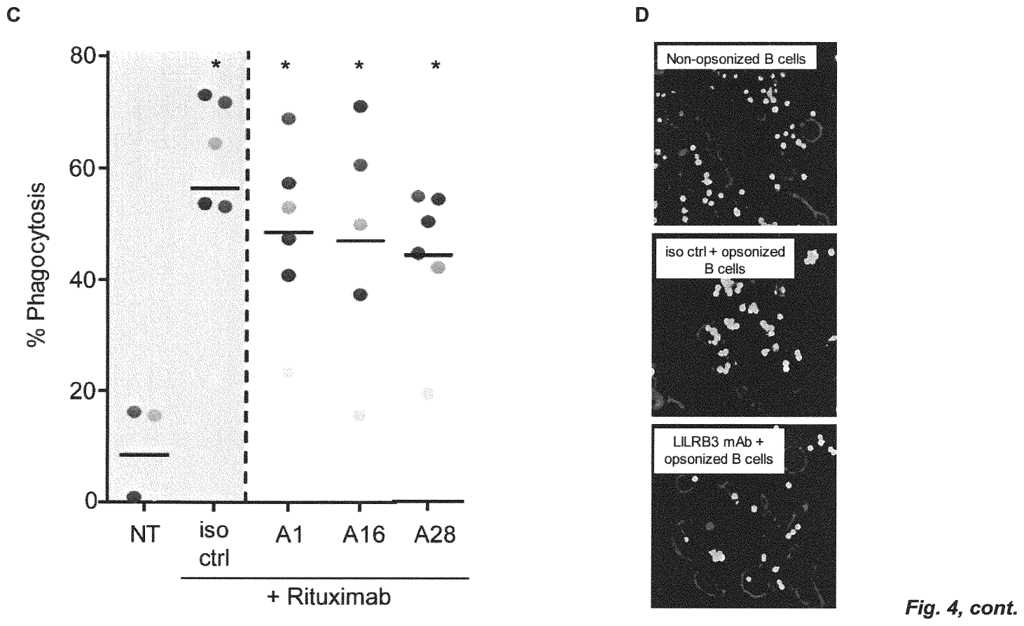

The above findings indicated that the LILRB3 mAb were able to agonize or antagonize LILRB3 to regulate T cell proliferation, likely through regulating APC function. Hence, as macrophages also express high levels of LILRB, and are known to be regulated by them (13), the effects of LILRB3 ligation on macrophage phagocytosis were studied. Staining with representative LILRB3 mAb confirmed high expression levels of LILRB3 on human MDMs (FIG. 4A). To assess any modulation of their effector function, CFSE-labelled primary CLL B cells were opsonized with anti-CD20 mAb (rituximab) and used as targets for macrophages in a phagocytosis assay (FIG. 4 B-C). The deglycosylated anti-LILRB3 clones significantly decreased the extent of phagocytosis (p<0.05 in all cases) (FIG. 4 C). These findings were further confirmed by confocal microscopy, showing lower number of CFSE+ target cells in LILRB3-treated macrophages, compared to isotype control (FIG. 4 D). These data demonstrate that the majority of LILRB3 mAb delivered inhibitory signals to reduce macrophage effector function. Importantly, the LILRB3 mAb were deglycosylated, capable of mediating only Fab-dependent effects without complications arising as a result of Fc:FcγR interactions (48).

### LILRB3 Ligation Induces Immune Tolerance in Humanized Mice

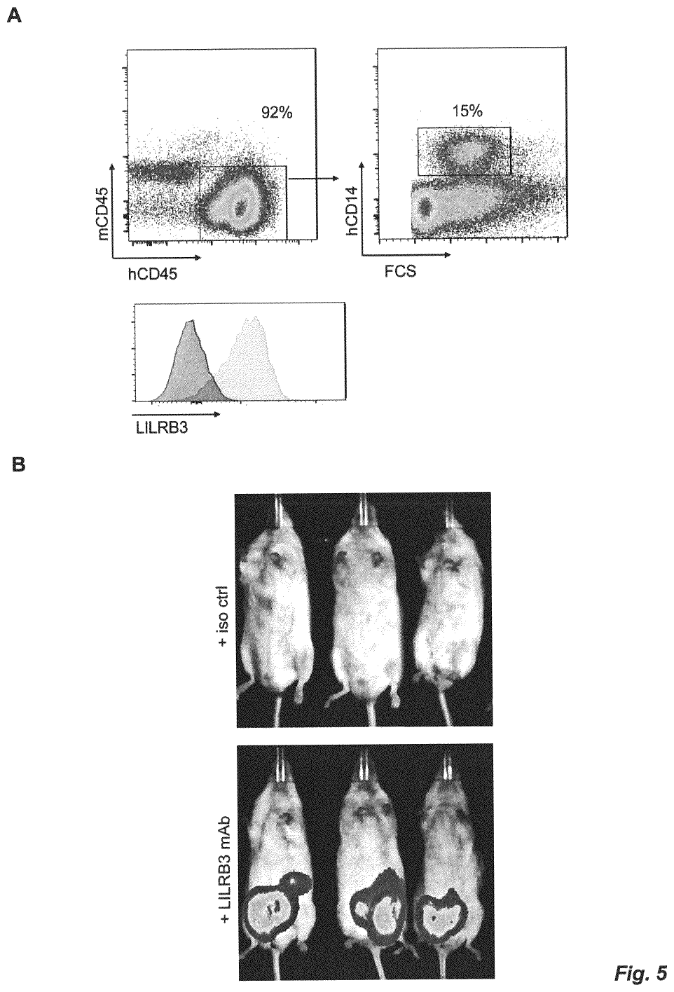

Given these data showing both adaptive (T cells) and innate (myeloid) activities can be suppressed following LILRB3 ligation, we next tested the possible effects of LILRB3 modulation in an allogeneic engraftment model using humanized mice (reconstituted with primary human HSC). Characterization of the humanized mice demonstrated that LILRB3 was expressed on the myeloid cells in a similar manner to human peripheral blood (FIG. 5 A). Allogeneic human lymphoma cells are readily rejected in humanized mice due to the HLA mismatch (data not shown; 49). To test the potential of LILRB3 ligation to suppress the allogeneic immune response, we pre-treated reconstituted adult humanized mice with an agonistic LILRB3 mAb (A1) and assessed the engraftment of allogeneic human ‘double-hit’ B cell lymphoma cells (31, 50) derived from unrelated donors. LILRB3 mAb treatment was able to induce a state of tolerance in the mice and led to a successful engraftment of human lymphoma cells (FIG. 5 B). LILRB3-treated tumor-bearing mice had to be humanely culled due to high tumor burden, whereas, isotype control-treated mice readily rejected the lymphoma cells without any morbidity. These observations further corroborate our in vitro functional assays and identify LILRB3 a key regulator of myeloid cells during an immune response.

### LILRB3 Ligation Leads to Transcriptional Modifications and M2-Skewing of Human APCs

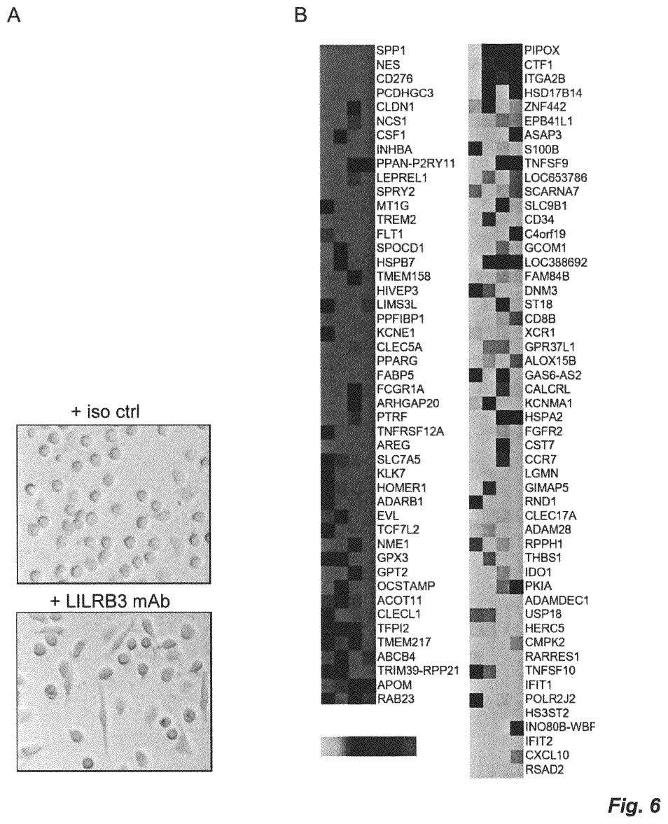

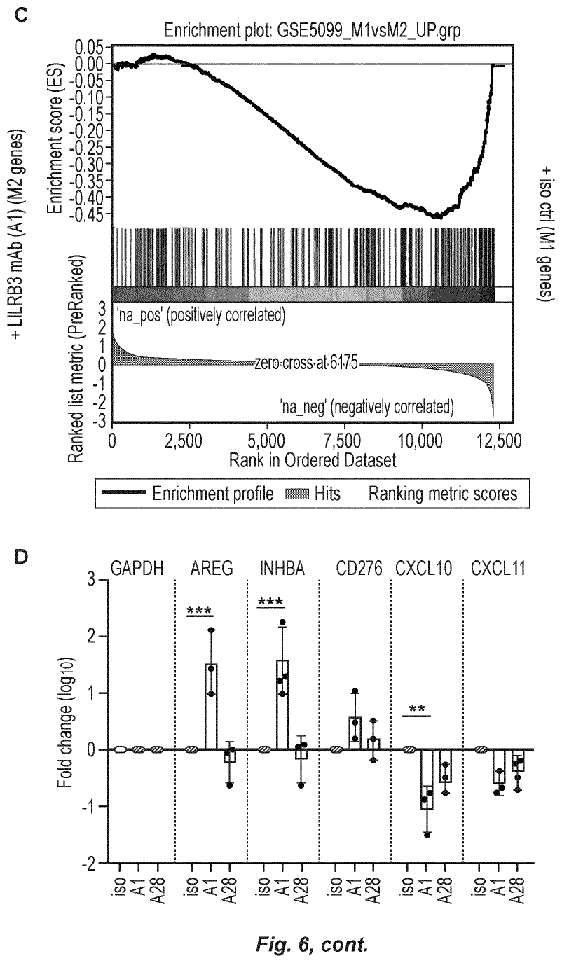

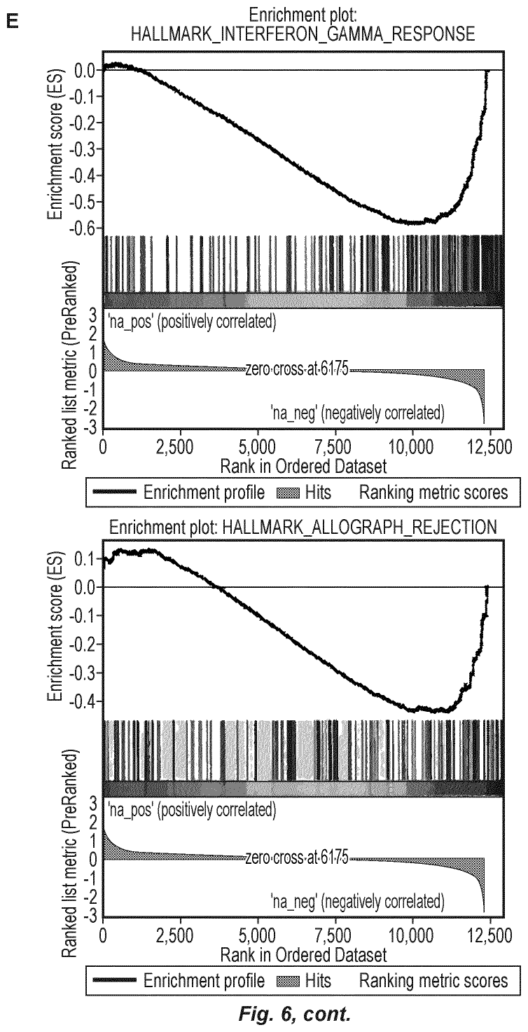

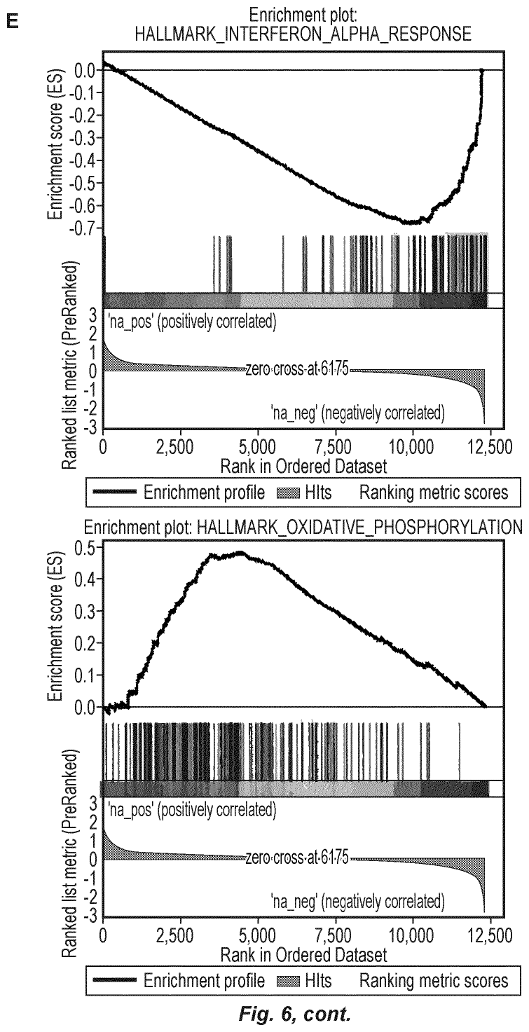

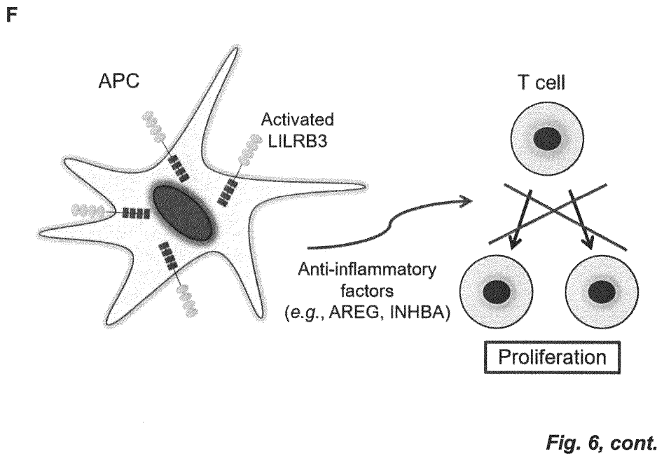

To investigate the pathways and factors involved in LILRB3-mediated immunosuppression we investigate the transcriptomic changes in primary APCs following LILRB3 engagement. Short-term (˜18 hour) in vitro treatment of isolated human peripheral CD14+ monocytes with agonistic LILRB3 mAb (A1) caused a dramatic shift in their phenotype (FIG. 6A), with the cells displaying an elongated morphology resembling “M2”, immunosuppressed IL4/IL-13 treated macrophages (51). RNAseq analysis revealed that ligation of LILRB3 on monocytes induced a signature resembling “M2”-skewed immunosuppressive macrophages (FIG. 6 B). Likewise, the expression of genes associated with “M1”-skewed immunostimulatory macrophages was downregulated in LILRB3 mAb-treated compared to isotype control-treated monocytes (FIG. 6 B-C). We confirmed these data by performing qPCR on a further 3 donors for a select number of differentially regulated genes (FIG. 6 D). Treatment of monocytes with a less/non-agonistic LILRB3 mAb (A28) did not affect monocyte phenotype and gene expression (data not shown and FIG. 6 C). Gene-set enrichment analysis (GSEA) showed a positive correlation with gene signatures reported for suppressive macrophages, e.g., oxidative phosphorylation (52). Conversely, LILRB3-ligated monocyte gene signatures negatively correlated with the gene signatures reported for inflammatory macrophages, e.g., IFN-γ and IFN-α responsive elements, as well as allograft rejection (FIG. 6 E). Taken together, these data confirm that LILRB3 activation results in significant phenotypic and transcriptional alterations in APCs, such as monocytes, leading to potent inhibition of downstream immune responses (FIG. 6 F).

### DISCUSSION

We previously demonstrated that ligation of LILRB1 on human monocytes induces a tolerogenic phenotype, subsequently hindering T cell responses (12, 53). In this study, we investigated another LILR family member, LILRB3, whose function is not yet determined, due to lack of suitable reagents and experimental systems. We, therefore, generated and characterized an extensive panel of fully human mAb with specificity for LILRB3. Staining of different leukocyte populations with the specific mAb confirmed that LILRB3 is mainly restricted to human myeloid cells (3). This was confirmed in several independent donors, suggesting that although these receptors are polymorphic (LILRB3 has at least ten variants (3, 54)), the antibodies recognize many if not all variants, which is important for the development of these reagents for therapeutic use. Subsequent analysis showed that the LILRB3 mAb displayed a range of affinities, all of which were in the nanomolar (nM) range with similar on-rates, but off-rates differing over three orders of magnitude. KD values that are of low nM range are generally considered to be viable drug candidates; rituximab, for example, has an 8 nM affinity for its target, CD20 (55). This suggests that the LILRB3 mAb generated here have potential as therapeutic agents. Some of the selected LILRB3 clones showed unexpected cross-reactivity to other human LILR-transfectants and were excluded from subsequent analysis. However, it should be noted that as LILR3B shares >95% sequence homology in its extracellular domain with LILRA6, LILRB3 mAb may well interact with LILRA6 if co-expressed (56). Furthermore, epitope mapping experiments revealed that the specific LILRB3 mAb were generated against two distinct epitopes, as they bound to either Ig-like extracellular domain two or four. None of the generated LILRB3 mAb bound to domains one or three, suggesting that these domains may not contain conserved unique epitopes.

The ability of the LILRB3 mAb to influence T cell responses was observed through either inhibition or enhancement of proliferation, indicating agonistic or antagonistic properties, respectively. Similar to LILRB1 (12, 13, 57), this is likely through an effect on APCs, as they are the only cells expressing LILRB3 in the culture. Unlike LILRB1 (42, 53, 583, 59), LILRB3 is not expressed on T cells, and can only affect T cell responses indirectly. Based on the LILRB3 expression pattern and frequency of the cells within the PBMC culture, monocytes represent the most likely cell type influenced. In support of this, agonistic LILRB3 mAb did not suppress T cell proliferation in the absence of monocytes. Binding epitopes influence the ability of mAb to modulate receptor function in many systems (29, 60) and so it was unsurprising to see LILRB3 mAb capable of opposing functions. The majority of LILRB3 mAb that bound to the second Ig-like domain of LILRB3 were able to inhibit T cell proliferation. Conversely, some clones that bound to domain four enhanced proliferation. However, a D4-binding mAb (A1) was one of the strongest inhibitors of proliferation and another D4-binding (A28) induced less inhibitory effect. Therefore, domain-specific epitopes do not seem to correlate directly with LILRB3 mAb-mediated effector cell functions.

Although the LILRB3 mAb showed variation in their ability to inhibit or enhance T cell proliferation, the majority of clones inhibited phagocytosis by macrophages or had no effect. This suggests that the majority of mAb are agonistic in this context, stimulating inhibitory signaling and suppressing effector function, akin to the inhibition of T cell responses.

Our observations demonstrating immunoinhibitory activities downstream of LILR3B were further confirmed in the reconstituted humanized mouse model. In this system, where LILRB3 is present only on the monocytic cells, ligation of LILRB3 with an agonistic LILRB3 mAb prior to engraftment of allogeneic lymphoma cells (31) induced tolerance in vivo and enabled subsequent tumor growth. This demonstrates the capacity of LILRB3 to exert profound immunosuppressive effects that may be exploited in therapeutic settings, such as autoimmunity and transplantation, where induction of immune tolerance will be beneficial. Although regarded as an orphan receptor, it has been suggested that LILRB3 associates with cytokeratin (CK)-associated proteins (exposed on necrotic cancer cells), angiopoietin-like protein 5 and bacteria, such as Staphylococcus aureus (S. aureus)(40, 61, 62). Therefore, our functional data suggest that certain pathogens (61) may be able to subvert immune responses by actively ligating LILRB3 during an active response.

To investigate the pathways and factors involved in LILRB3-mediated immunosuppression, we investigated the transcriptomic changes in isolated peripheral myeloid cells following LILRB3 activation. Over one hundred genes were differentially regulated in primary human monocytes following LILRB3 ligation, some of which are known to be modulated in M2 macrophages and TAMs. Amphiregulin was among the genes whose expression was significantly upregulated in LILRB3-ligated monocytes. Amphiregulin is an epidermal growth factor-like growth factor, responsible for inducing tolerance and immunosuppression, via various mechanisms including enhancement of Treg activity (63). Furthermore, amphiregulin is overexpressed in tumor-associated DCs (64) and suppressive/M2 macrophages (65) and has been suggested to play a crucial role in immunosuppression and cancer progression (66). Such LILRB3-inducible factors may be responsible for the suppression observed in our T cell assays. Our ongoing efforts aim to test this and fully understand the mechanisms responsible for LILRB3-mediated suppression of myeloid cells. A recent study investigating the mode of action of Glatiramer acetate (Copaxone), a peptide-based drug used to treat patients with the relapsing-remitting form of multiple sclerosis that ameliorates autoimmunity, identified LILRB2 and LILRB3 as potential ligands (67). Targeting human LILRB2 with antagonistic mAb on human myeloid cells is able to promote their pro-inflammatory activity and enhance antitumor responses in vivo (13). Furthermore, recent data by Zhang and colleagues suggests that LILRB4 signaling in leukemia cells mediates T cell suppression of supports tumor cell dissemination to distal organs (68). These data further support our findings, demonstrating that activation of human LILRBs induce immunosuppression via reprogramming myeloid cells (i.e., reducing M1-like maturation and promoting MDSC-suppressive function).

Together the findings presented here show that LILRB3 activation on primary human myeloid cells exerts potent immunoinhibitory functions and that LILRB3-specific mAb are potentially powerful immunomodulatory agents, with a broad application ranging from transplantation to autoimmunity to inflammatory disorders, and beyond.

### REFERENCES

1. K. J. Anderson, R. L. Allen, Regulation of T-cell immunity by leucocyte immunoglobulin-like receptors: innate immune receptors for self on antigen-presenting cells. Immunology 127, 8-17 (2009).
2. W. van der Touw, H. M. Chen, P. Y. Pan, S. H. Chen, LILRB receptor-mediated regulation of myeloid cell maturation and function. Cancer Immunol Immunother 66, 1079-1087 (2017).
3. M. Colonna et al., A common inhibitory receptor for major histocompatibility complex class I molecules on human lymphoid and myelomonocytic cells. J Exp Med 186, 1809-1818 (1997).
4. L. Borges, M. L. Hsu, N. Fanger, M. Kubin, D. Cosman, A family of human lymphoid and myeloid Ig-like receptors, some of which bind to MHC class I molecules. J Immunol 159, 5192-5196 (1997).
5. H. Nakajima, J. Samaridis, L. Angman, M. Colonna, Human myeloid cells express an activating ILT receptor (ILT1) that associates with Fc receptor gamma-chain. J Immunol 162, 5-8 (1999).
6. C. C. Chang et al., Polymorphism and linkage disequilibrium of immunoglobulin-like transcript 3 gene. Hum Immunol 69, 284-290 (2008).
7. F. W. Velten, K. Duperrier, J. Bohlender, P. Metharom, S. Goerdt, A gene signature of inhibitory MHC receptors identifies a BDCA3(+) subset of IL-10-induced dendritic cells with reduced allostimulatory capacity in vitro. Eur J Immunol 34, 2800-2811 (2004).
8. C. C. Chang et al., Tolerization of dendritic cells by T(S) cells: the crucial role of inhibitory receptors ILT3 and ILT4. Nat Immunol 3, 237-243 (2002).
9. M. Beyer et al., High-resolution transcriptome of human macrophages. PLoS One 7, e45466 (2012).
10. J. S. Manavalan et al., High expression of ILT3 and ILT4 is a general feature of tolerogenic dendritic cells. Transpl Immunol 11, 245-258 (2003).
11. N. A. Fanger et al., The MHC class I binding proteins LIR-1 and LIR-2 inhibit Fc receptor-mediated signaling in monocytes. Eur J Immunol 28, 3423-3434 (1998).
12. N. T. Young et al., The inhibitory receptor LILRB1 modulates the differentiation and regulatory potential of human dendritic cells. Blood 111, 3090-3096 (2008).
13. A. A. Barkal et al., Engagement of MHC class I by the inhibitory receptor LILRB1 suppresses macrophages and is a target of cancer immunotherapy. Nat Immunol 19, 76-84 (2018).
14. M. K. Rochat et al., Maternal vitamin D intake during pregnancy increases gene expression of ILT3 and ILT4 in cord blood. Clin Exp Allergy 40, 786-794 (2010).
15. M. Brenk et al., Tryptophan deprivation induces inhibitory receptors ILT3 and ILT4 on dendritic cells favoring the induction of human CD4+CD25+ Foxp3+ T regulatory cells. J Immunol 183, 145-154 (2009).
16. M. G. Petroff, P. Sedlmayr, D. Azzola, J. S. Hunt, Decidual macrophages are potentially susceptible to inhibition by class Ia and class Ib HLA molecules. J Reprod Immunol 56, 3-17 (2002).
17. R. Apps, L. Gardner, A. M. Sharkey, N. Holmes, A. Moffett, A homodimeric complex of HLA-G on normal trophoblast cells modulates antigen-presenting cells via LILRB1. Eur J Immunol 37, 1924-1937 (2007).
18. L. Lombardelli et al., HLA-G5 induces IL-4 secretion critical for successful pregnancy through differential expression of ILT2 receptor on decidual CD4(+) T cells and macrophages. J Immunol 191, 3651-3662 (2013).
19. B. Favier, J. Lemaoult, E. Lesport, E. D. Carosella, ILT2/HLA-G interaction impairs NK-cell functions through the inhibition of the late but not the early events of the NK-cell activating synapse. FASEB J 24, 689-699 (2010).
20. S. Endo, Y. Sakamoto, E. Kobayashi, A. Nakamura, T. Takai, Regulation of cytotoxic T lymphocyte triggering by PIR-B on dendritic cells. Proceedings of the National Academy of Sciences of the United States of America 105, 14515-14520 (2008).
21. S. Pereira, H. Zhang, T. Takai, C. A. Lowell, The inhibitory receptor PIR-B negatively regulates neutrophil and macrophage integrin signaling. J Immunol 173, 5757-5765 (2004).
22. N. S. Wilson et al., An Fcgamma receptor-dependent mechanism drives antibody-mediated target-receptor signaling in cancer cells. Cancer Cell 19, 101-113 (2011).
23. W. Zhang, S. Liang, J. Wu, A. Horuzsko, Human Inhibitory Receptor ILT2 Amplifies CD11b(+)Gr1(+) Myeloid-Derived Suppressor Cells that Promote LongTerm Survival of Allografts. Transplantation 86, 1125-1134 (2008).
24. J. Wu et al., Isoforms of human leukocyte antigen-G and their inhibitory receptors in human kidney allograft acceptancet. Human Immunology 70, 988-994 (2009).
25. A. Roghanian et al., Cyclophosphamide Enhances Cancer Antibody Immunotherapy in the Resistant Bone Marrow Niche by Modulating Macrophage FcgammaR Expression. Cancer Immunol Res 7, 1876-1890 (2019).
26. E. Soderlind et al., Recombining germline-derived CDR sequences for creating diverse single-framework antibody libraries. Nature biotechnology 18, 852-856 (2000).
27. A. Roghanian et al., Antagonistic human FcgammaRIIB (CD32B) antibodies have anti-tumor activity and overcome resistance to antibody therapy in vivo. Cancer Cell 27, 473-488 (2015).
28. N. Olsson et al., Proteomic Analysis and Discovery Using Affinity Proteomics and Mass Spectrometry. Mol Cell Proteomics 10, (2011).
29. L. N. Dahal, A. Roghanian, S. A. Beers, M. S. Cragg, FcgammaR requirements leading to successful immunotherapy. Immunological reviews 268, 104-122 (2015).
30. A. Roghanian et al., Filament-associated TSGA10 protein is expressed in professional antigen presenting cells and interacts with vimentin. Cell Immunol 265, 120-126 (2010).
31. I. Leskov et al., Rapid generation of human B-cell lymphomas via combined expression of Myc and Bcl2 and their use as a preclinical model for biological therapies. Oncogene 32, 1066-1072 (2013).
32. B. Langmead, C. Trapnell, M. Pop, S. L. Salzberg, Ultrafast and memory-efficient alignment of short DNA sequences to the human genome. Genome Biol 10, R25 (2009).
33. B. Li, C. N. Dewey, RSEM: accurate transcript quantification from RNA-Seq data with or without a reference genome. BMC Bioinformatics 12, 323 (2011).
34. M. D. Robinson, D. J. McCarthy, G. K. Smyth, edgeR: a Bioconductor package for differential expression analysis of digital gene expression data. Bioinformatics 26, 139-140 (2010).
35. D. W. Huang et al., DAVID Bioinformatics Resources: expanded annotation database and novel algorithms to better extract biology from large gene lists. Nucleic Acids Res 35, W169-175 (2007).
36. A. Subramanian et al., Gene set enrichment analysis: a knowledge-based approach for interpreting genome-wide expression profiles. Proc Natl Acad Sci USA 102, 15545-15550 (2005).
37. F. O. Martinez, S. Gordon, M. Locati, A. Mantovani, Transcriptional profiling of the human monocyte-to-macrophage differentiation and polarization: new molecules and patterns of gene expression. J Immunol 177, 7303-7311 (2006).
38. A. I. Saeed et al., TM4: a free, open-source system for microarray data management and analysis. Biotechniques 34, 374-378 (2003).
39. N. Tedla et al., Activation of human eosinophils through leukocyte immunoglobulin-like receptor 7. Proceedings of the National Academy of Sciences of the United States of America 100, 1174-1179 (2003).
40. D. C. Jones et al., Allele-specific recognition by LILRB3 and LILRA6 of a cytokeratin 8-associated ligand on necrotic glandular epithelial cells. Oncotarget 7, 15618-15631 (2016).
41. D. Saverino et al., The CD85/LIR-1/ILT2 inhibitory receptor is expressed by all human T lymphocytes and down-regulates their functions. J Immunol 165, 3742-3755 (2000).
42. M. Shiroishi et al., Human inhibitory receptors Ig-like transcript 2 (ILT2) and ILT4 compete with CD8 for MHC class I binding and bind preferentially to HLA-G. Proceedings of the National Academy of Sciences of the United States of America 100, 8856-8861 (2003).
43. C. C. Chang et al., BCL6 Is Required for Differentiation of Ig-Like Transcript 3-Fc-Induced CD8(+) T Suppressor Cells. J Immunol 185, 5714-5722 (2010).
44. R. A. Clynes, T. L. Towers, L. G. Presta, J. V. Ravetch, Inhibitory Fc receptors modulate in vivo cytotoxicity against tumor targets. Nature medicine 6, 443-446 (2000).
45. C. S. Lee et al., Expression of the inhibitory Fc gamma receptor IIB (FCGR2B, CD32B) on follicular lymphoma cells lowers the response rate to rituximab monotherapy (SAKK 35/98). British journal of haematology 168, 145-148 (2015).
46. C. E. Hargreaves et al., Fcgamma receptors: genetic variation, function, and disease. Immunological reviews 268, 6-24 (2015).
47. Y. Kaneko, F. Nimmerjahn, E. V. Ravetch, Anti-inflammatory activity of immunoglobulin G resulting from Fc sialylation. Science 313, 670-673 (2006).
48. P. M. Hogarth, G. A. Pietersz, Fc receptor-targeted therapies for the treatment of inflammation, cancer and beyond. Nat Rev Drug Discov 11, 311-331 (2012).
49. A. Roghanian et al., Cyclophosphamide Enhances Cancer Antibody Immunotherapy in the Resistant Bone Marrow Niche by Modulating Macrophage FcgammaR Expression. Cancer Immunol Res, (2019).
50. C. P. Pallasch et al., Sensitizing protective tumor microenvironments to antibody-mediated therapy. Cell 156, 590-602 (2014).
51. F. Y. McWhorter, T. Wang, P. Nguyen, T. Chung, W. F. Liu, Modulation of macrophage phenotype by cell shape. Proceedings of the National Academy of Sciences of the United States of America 110, 17253-17258 (2013).
52. S. Galvan-Pena, L. A. O'Neill, Metabolic reprograming in macrophage polarization. Front Immunol 5, 420 (2014).
53. R. C. Khanolkar et al., Leukocyte Ig-Like receptor B1 restrains dendritic cell function through increased expression of the NF-kappaB regulator ABIN1/TNIP1. J Leukoc Biol, (2016).
54. A. A. Bashirova et al., Diversity of the human LILRB3/A6 locus encoding a myeloid inhibitory and activating receptor pair. Immunogenetics 66, 1-8 (2014).
55. M. D. Pescovitz, Rituximab, an anti-cd20 monoclonal antibody: history and mechanism of action. Am J Transplant 6, 859-866 (2006).
56. M. R. Lopez-Alvarez, D. C. Jones, W. Jiang, J. A. Traherne, J. Trowsdale, Copy number and nucleotide variation of the LILR family of myelomonocytic cell activating and inhibitory receptors. Immunogenetics 66, 73-83 (2014).
57. C. S. Wagner et al., Human cytomegalovirus-derived protein UL18 alters the phenotype and function of monocyte-derived dendritic cells. J Leukoc Biol 83, 56-63 (2008).
58. J. Dietrich, M. Cella, M. Colonna, Ig-like transcript 2 (ILT2)/leukocyte Ig-like receptor 1 (LIR1) inhibits TCR signaling and actin cytoskeleton reorganization. J Immunol 166, 2514-2521 (2001).
59. F. Ketroussi et al., Lymphocyte Cell-Cycle Inhibition by HLA-G Is Mediated by Phosphatase SHP-2 and Acts on the mTOR Pathway. Plos One 6, (2011).
60. X. Yu et al., Complex Interplay between Epitope Specificity and Isotype Dictates the Biological Activity of Anti-human CD40 Antibodies. Cancer Cell 33, 664-675 e664 (2018).
61. M. Nakayama et al., Paired Ig-like receptors bind to bacteria and shape TLR-mediated cytokine production. J Immunol 178, 4250-4259 (2007).
62. J. Zheng et al., Inhibitory receptors bind ANGPTLs and support blood stem cells and leukaemia development. Nature 485, 656-660 (2012).
63. D. M. W. Zaiss, W. C. Gause, L. C. Osborne, D. Artis, Emerging functions of amphiregulin in orchestrating immunity, inflammation, and tissue repair. Immunity 42, 216-226 (2015).
64. Y. L. Hsu et al., Lung tumor-associated dendritic cell-derived amphiregulin increased cancer progression. J Immunol 187, 1733-1744 (2011).
65. P. Vlaicu et al., Monocytes/macrophages support mammary tumor invasivity by co-secreting lineage-specific EGFR ligands and a STAT3 activator. BMC Cancer 13, 197 (2013).
66. B. Busser, L. Sancey, E. Brambilla, J. L. Coll, A. Hurbin, The multiple roles of amphiregulin in human cancer. Biochim Biophys Acta 1816, 119-131 (2011).
67. W. van der Touw et al., Glatiramer Acetate Enhances Myeloid-Derived Suppressor Cell Function via Recognition of Paired Ig-like Receptor B. J Immunol 201, 1727-1734 (2018).
68. M. Deng et al., LILRB4 signalling in leukaemia cells mediates T cell suppression and tumour infiltration. Nature 562, 605-609 (2018).

1. A method for the treatment of graft rejection, an autoimmune disorder and/or an inflammatory disorder in a patient comprising administering to the patient an antibody molecule that binds specifically to LILRB3 (ILT5).

2. A method according to claim 1, wherein said antibody molecule is an agonistic antibody molecule.

3. An antibody molecule that binds specifically to LILRB3 (ILT5), wherein the antibody molecule is selected from the group consisting of antibody molecules comprising 1-6 of the CDRs VH-CDR1, VH-CDR2, VH-CDR3, VL-CDR1, VL-CDR2 and VL-CDR3,
wherein VH-CDR1, if present, is selected from the group consisting of SEQ. ID. NOs: 1, 9, 17 and 25;
wherein VH-CDR2, if present, is selected from the group consisting of SEQ. ID. NOs: 2, 10, 18 and 26;
wherein VH-CDR3, if present, is selected from the group consisting of SEQ. ID. NOs: 3, and 19 and 27;
wherein VL-CDR1, if present, is selected from the group consisting of SEQ. ID. NOs: 4, 12, 20 and 28;
wherein VL-CDR2, if present, is selected from the group consisting of SEQ. ID. NOs: 5, 13, 21 and 29; and
wherein VL-CDR3, if present, is selected from the group consisting of SEQ. ID. NOs: 6, 14, 22 and 30.

4. An antibody molecule according to claim 3, wherein the antibody molecule comprises a variable heavy chain (VH) comprising the following CDRs:
(i) SEQ. ID. NO: 1, SEQ. ID. NO: 2 and SEQ. ID. NO: 3; or
(ii) SEQ. ID. NO: 9, SEQ. ID. NO: 10 and SEQ. ID. NO: 11; or
(iii) SEQ. ID. NO: 17, SEQ. ID. NO: 18 and SEQ. ID. NO: 19; or
(iv) SEQ. ID. NO: 25, SEQ. ID. NO: 26 and SEQ. ID. NO: 27
and/or wherein the antibody molecule comprises a variable light chain (VL) comprising the following CDRs:
(v) SEQ. ID. NO: 4, SEQ. ID. NO: 5 and SEQ. ID. NO: 6; or
(vi) SEQ. ID. NO: 12, SEQ. ID. NO: 13 and SEQ. ID. NO: 14; or
(vii) SEQ. ID. NO: 20, SEQ. ID. NO: 21 and SEQ. ID. NO: 22; or
(viii) SEQ. ID. NO: 28, SEQ. ID. NO: 29 and SEQ. ID. NO: 30.

5. An antibody molecule according to claim, wherein the antibody molecule comprises a variable heavy chain (VH) amino acid sequence selected from the group consisting of SEQ. ID. NOs 7, 15, 23 and 31; and/or wherein the antibody molecule comprises a variable light chain (VL) amino acid sequence selected from the group consisting of SEQ. ID. NOs: 8, 16, 24 and 32.

6. An antibody molecule according to claim 3, wherein the antibody molecule is an agonistic antibody molecule.

7. A method according to claim 1, or an antibody molecule according to any one of the claims 3-6, wherein the antibody molecule is selected from the group consisting of a wild-type or Fc engineered human IgG antibody molecule, a humanized IgG antibody molecule, and an IgG antibody molecule of human origin.

8. A method according to claim 7 or an antibody molecule according to claim 7, wherein the antibody molecule is a human IgG1, IgG2 or IgG4 antibody.

9. An antibody molecule for use according to claim 1 or 2, or an antibody molecule according to any one of the claims 3-8, wherein the antibody molecule is a monoclonal antibody.

10. A method according to claim 1, wherein the antibody is selected from the group consisting of antibody molecules comprising 1-6 of the CDRs VH-CDR1, VH-CDR2, VH-CDR3, VL-CDR1, VL-CDR2 and VL-CDR3,
wherein VH-CDR1, if present, is selected from the group consisting of SEQ. ID. NOs: 1, 9, 17 and 25;
wherein VH-CDR2, if present, is selected from the group consisting of SEQ. ID. NOs: 2, 10, 18 and 26;
wherein VH-CDR3, if present, is selected from the group consisting of SEQ. ID. NOs: 3, and 19 and 27;
wherein VL-CDR1, if present, is selected from the group consisting of SEQ. ID. NOs: 4, 12, 20 and 28;
wherein VL-CDR2, if present, is selected from the group consisting of SEQ. ID. NOs: 5, 13, 21 and 29; and
wherein VL-CDR3, if present, is selected from the group consisting of SEQ. ID. NOs: 6, 14, 22 and 30.

11. The method according to claim 1, wherein the antibody molecule is an antibody molecule that is capable of competing for binding to LILRB3 (ILT5) with an antibody molecule selected from the group consisting of antibody molecules comprising 1-6 of the CDRs VH-CDR1, VH-CDR2, VH-CDR3, VL-CDR1, VL-CDR2 and VL-CDR3,
wherein VH-CDR1, if present, is selected from the group consisting of SEQ. ID. NOs: 1, 9, 17 and 25;
wherein VH-CDR2, if present, is selected from the group consisting of SEQ. ID. NOs: 2, 10, 18 and 26;
wherein VH-CDR3, if present, is selected from the group consisting of SEQ. ID. NOs: 3, and 19 and 27;
wherein VL-CDR1, if present, is selected from the group consisting of SEQ. ID. NOs: 4, 12, 20 and 28;
wherein VL-CDR2, if present, is selected from the group consisting of SEQ. ID. NOs: 5, 13, 21 and 29; and
wherein VL-CDR3, if present, is selected from the group consisting of SEQ. ID. NOs: 6, 14, 22 and 30.

12. An isolated nucleotide sequence encoding an antibody molecule as defined in claim 3.

13. A plasmid comprising a nucleotide sequence as defined in claim 12.

14. A cell comprising a nucleotide sequence as defined in claim 12.

15. A method for the treatment of a graft rejection, an autoimmune disorder and/or an inflammatory disorder in a patient comprising administering to the patient nucleotide sequence according to claim 12.

16-17. (canceled)

18. A pharmaceutical composition comprising or consisting of an antibody molecule as defined in claim 3, optionally a pharmaceutically acceptable diluent, carrier, vehicle and/or excipient.

19. A pharmaceutical composition according to claim 18, for use in the treatment of graft rejection, an autoimmune disorder and/or an inflammatory disorder.

20. (canceled)

21. The method according to claim 1, wherein the antibody molecule is an agonistic antibody molecule binding specifically to LILRB3 (ILT5).

22. A method for treatment of graft rejection, an autoimmune disorder and/or an inflammatory disorder in a patient comprising administering to the patient a therapeutically effective amount of a nucleotide sequence according to claim 12.

23. A pharmaceutical composition comprising or consisting of a nucleotide sequence according to claim 12, and optionally a pharmaceutically acceptable diluent, carrier, vehicle and/or excipient.
